(19) United States
(12) Patent Application Publication
(10) Pub. No.: US 2020/0282024 A1
(43) Pub. Date: Sep. 10, 2020

(54) FORMULATIONS FOR COMPOUND DELIVERY

(71) Applicant: Ra Pharmaceuticals, Inc., Cambridge, MA (US)

(72) Inventors: Richard Lee Fahrner, Cambridge, MA (US); Christian Winthrop Phillips, Cambridge, MA (US)

(21) Appl. No.: 16/646,263

(22) PCT Filed: Sep. 11, 2018
(86) PCT No.: PCT/US2018/050317

§ 371 Date: Mar. 11, 2020

Related U.S. Application Data
(60) Provisional application No. 62/556,656, filed on Sep. 11, 2017.

(51) Int. Cl. A61K 38/46; A61K 47/14; A61K 47/10

(52) U.S. Cl. CPC A61K 38/465; A61K 47/10; A61K 47/14

(57) ABSTRACT

The present disclosure provides formulations for sustained release of therapeutic agents. Included are formulations incorporating a mixture of acylglycerols. Also included are methods of delivering therapeutic agents to subjects using sustained release formulations and methods of treating complement-related indications using formulations disclosed.

### CROSS REFERENCE TO RELATED APPLICATIONS

This application claims priority to U.S. Provisional Application No. 62/556,656 filed on Sep. 11, 2017 entitled Formulations for Compound Delivery, the contents of which are herein incorporated by reference in their entirety.

### SEQUENCE LISTING

The present application is being filed along with a Sequence Listing in electronic format. The Sequence Listing file, entitled 2011_1014PCT_SL.txt, was created on Sep. 5, 2018 and is 1,080 bytes in size. The information in electronic format of the Sequence Listing is incorporated herein by reference in its entirety.

### BACKGROUND

A therapeutic window is defined for pharmacologically active agents as a range, generally serum or plasma levels, where a beneficial biological effect is observed. At levels below the window, limited or decreased physiological benefit is observed. At drug levels above the window, a diminished beneficial pharmacological effect can be observed and/or potential toxic effects may occur. Thus, it is not only desirable but often required that drug levels remain within the therapeutic window to achieve the maximal physiological effect. When the rate of drug clearance is high and/or the therapeutic window is narrow, more frequent dose administration is necessary to maintain drug concentrations within the therapeutic window.

Achieving stable drug concentrations and thereby disease control can be particularly difficult in cases of poor patient compliance. There remains a need for single administration formulations capable of providing concentrations of therapeutic agents within therapeutic windows over longer durations. The present disclosure addresses this need with formulations for controlled delivery of therapeutic agents.

### SUMMARY OF THE INVENTION

In some embodiments, the present disclosure provides a sustained release formulation that includes a therapeutic agent and a mixture of acylglycerols. The mixture of acylglycerols may include from about 40% to about 70% monoglycerides; from about 0% to about 60% diglycerides; and from about 0% to about 60% triglycerides. At least one acylglycerol from the mixture of acylglycerols may include at least one fatty acid selected from one or more of a long chain fatty acid and a medium chain fatty acid. At least one acylglycerol from the mixture of acylglycerols may include an unsaturated fatty acid. At least one acylglycerol from the mixture of acylglycerols may include a saturated fatty acid. At least one acylglycerol from the mixture of acylglycerols may include a long chain fatty acid selected from one or more of linoleic acid and oleic acid. At least one acylglycerol from the mixture of acylglycerols may include a medium chain fatty acid selected from one or more of capric acid and caprylic acid. The mixture of acylglycerols may include about 60% monoglycerides. The monoglycerides may include linoleic acid. The mixture of acylglycerols may include about 20% diglycerides. The mixture of acylglycerols may include less than 20% diglycerides. The diglycerides may include capric acid. The mixture of acylglycerols may include about 20% triglycerides. The mixture of acylglycerols may include less than 20% triglycerides. The triglycerides may include capric acid. The formulation may include about 60% monolinolein. The formulation may include about 20% dicaprylin. The formulation may include about 20% tricaprylin. The formulation may include at least one excipient. The excipient may include phosphate buffered saline. The excipient may include sodium deoxycholate. The formulation may include propylene glycol. The formulation may include a surfactant. The formulation may include a peptide or peptidomimetic. The therapeutic agent may be a complement inhibitor. The complement inhibitor may be a C5 inhibitor. The C5 inhibitor may be R5000.

In some embodiments, the present disclosure provides a method of delivering a therapeutic agent to a subject by preparing a formulation described herein and administering the formulation to the subject. The formulation may be a low viscosity formulation prior to administration. The formulation may become highly viscous upon administration to the subject. The formulation may form a highly viscous non-lamellar liquid crystalline phase upon administration to the subject. The formulation may become highly viscous upon contact with an aqueous bodily fluid. The therapeutic agent may be continuously released from the formulation over an extended period of time after administration. The formulation may be administered parenterally. The formulation may be administered intravitreally, intrathecally, subdurally, epidurally, intraperitoneally, intramuscularly, subcutaneously, or intradermally.

Methods of the present disclosure include a method of inhibiting complement activity in a subject by administering a formulation described herein to a subject, wherein the formulation includes R5000 as a therapeutic agent. R5000 may be present in the formulation at a concentration of from about 10 mg/ml to about 500 mg/ml. The formulation may be administered to the subject at a dose sufficient to provide from about 1 mg/kg to about 500 mg/kg of R5000.

In some embodiments, the present disclosure provides a method of treating a complement-related indication in a subject by administering a formulation described herein, wherein the formulation includes R5000 as a therapeutic agent. R5000 may be present in the formulation at a concentration of from about 10 mg/ml to about 500 mg/ml. The formulation may be administered to the subject at a dose sufficient to provide from about 1 mg/kg to about 500 mg/kg of R5000.

### BRIEF DESCRIPTION OF THE FIGURES

The foregoing and other objects, features and advantages will be apparent from the following description of particular embodiments of the invention, as well as the accompanying drawings illustrating the principles of various embodiments of the invention.

FIG. 1 is a graph showing changes in R5000 concentration over time in blood samples from a rat single injection pharmacokinetic model using different R5000 formulations.

FIG. 2 is a graph showing R5000 concentration and activity over time in blood samples from a monkey single injection pharmacokinetic-pharmacodynamic model using different R5000 formulations.

FIG. 3A is a graph showing the percent of drug released from R5000 formulations in an in vitro release assay.

FIG. 3B is a graph showing the amount of drug released from R5000 formulations in an in vitro release assay.

FIG. 4A is a graph showing the percent of drug released from R5000 formulations in an in vitro release assay.

FIG. 4B is a graph showing R5000 concentration and activity over time in blood samples from a monkey single injection pharmacokinetic-pharmacodynamic model using different R5000 formulations.

FIG. 5 is a graph showing R5000 concentration and activity over time in blood samples from a monkey single injection pharmacokinetic-pharmacodynamic model using different R5000 formulations.

FIG. 6A is a graph showing the percent of drug released from R5000 formulations in an in vitro release assay.

FIG. 6B is a graph showing R5000 concentration and activity over time in blood samples from a monkey single injection pharmacokinetic-pharmacodynamic model using different R5000 formulations.

FIG. 6C is a graph showing changes in R5000 concentration over time in blood samples from a rat single injection pharmacokinetic model using different R5000 formulations.

FIG. 7A is a graph showing the percent of drug released from R5000 formulations in an in vitro release assay.

FIG. 7B is a graph showing R5000 concentration and activity over time in blood samples from a monkey single injection pharmacokinetic-pharmacodynamic model using different R5000 formulations.

FIG. 8A is a graph showing the percent of drug released from R5000 formulations in an in vitro release assay.

FIG. 8B is a graph showing changes in R5000 concentration over time in blood samples from a rat single injection pharmacokinetic model using different R5000 formulations.

FIG. 8C is a graph showing R5000 concentration and activity over time in blood samples from a monkey single injection pharmacokinetic-pharmacodynamic model using different R5000 formulations.

FIG. 9A is a graph showing the percent of drug released from R5000 formulations in an in vitro release assay.

FIG. 9B is a graph showing the percent of drug released from R5000 formulations in an in vitro release assay.

FIG. 10A is a graph showing the percent of drug released from R5000 formulations in an in vitro release assay.

FIG. 10B is a graph showing changes in R5000 concentration over time in blood samples from a rat single injection pharmacokinetic model using different R5000 formulations.

FIG. 11 is a graph showing R5000 concentration and activity over time in blood samples from a monkey single injection pharmacokinetic-pharmacodynamic model using different R5000 formulations.

FIG. 12A is a graph showing the percent of drug released from R5000 formulations in an in vitro release assay.

FIG. 12B is a graph showing R5000 concentration and activity over time in blood samples from a monkey single injection pharmacokinetic-pharmacodynamic model using different R5000 formulations.

FIG. 13A is a graph showing the percent of drug released from R5000 formulations in an in vitro release assay.

FIG. 13B is a graph showing R5000 concentration and activity over time in blood samples from a monkey single injection pharmacokinetic-pharmacodynamic model using different R5000 formulations.

### DETAILED DESCRIPTION

Embodiments of the present disclosure include formulations for delivery of therapeutic agents. In some embodiments, low viscosity injectable formulations are provided that may be used for sustained release of therapeutic agents, including polypeptide inhibitors. Formulations presented herein may provide for the dissolution of therapeutic agents in mixtures of mono-, di-, and tri-glycerides of long- and/or medium-chain fatty acids. Upon injection and absorption of fluids, these formulations may transform into highly viscous non-lamellar liquid crystalline gel phases. Due to the high viscosity and hydrophobic nature of formulations in liquid crystalline states, therapeutic agent release rate into circulation may be slowed, in some instances for a sustained period. The release rate and release profile (such as Cₘₐₓ) of some formulations are tailored by varying the ratio of mono-, di-, and tri-glycerides contained within the lipid excipient and/or by using combinations of glycerides with various chain lengths and degrees of unsaturation. Formulations may be optimized by incorporating one or more of lipids with different head-groups, co-solvents, surfactants, antioxidants, and different salt forms of therapeutic agents being released.

### Therapeutic Agents

In some embodiments, the present disclosure provides formulations for administration and delivery of therapeutic agents. Therapeutic agents may include, but are not limited to, natural products, synthetic products, combinations of natural and synthetic products, small molecules, macromolecules, nucleic acids, aptamers, proteins, and polypeptides. Any amino acid-based molecule (natural or unnatural) may be termed a “polypeptide” and this term embraces “peptides,” “peptidomimetics,” and “proteins.” “Peptides” are traditionally considered to range in size from about 4 to about 50 amino acids. Polypeptides larger than about 50 amino acids are generally termed “proteins.” A “peptidomimetic” or “polypeptide mimetic” is a polypeptide in which the molecule contains structural elements that are not found in natural polypeptides (i.e., polypeptides comprised of only the 20 proteinogenic amino acids).

In some embodiments, therapeutic agents of the present disclosure may include complement inhibitors. Complement inhibitors are therapeutic agents that inhibit complement activity. As used herein, “complement activity” includes the activation of the complement cascade; the formation of cleavage products from a complement component such as C3 or C5; the assembly of downstream complexes following a cleavage event; or any process or event attendant to, or resulting from, the cleavage of a complement component, e.g., C3 or C5. Complement inhibitors may include C5 inhibitors that block complement activation at the level of complement component C5. C5 inhibitors may bind C5 and prevent its cleavage, by C5 convertase, into the cleavage products C5a and C5b. As used herein, “complement component C5” or “C5” is defined as a complex which is cleaved by C5 convertase into at least the cleavage products C5a and C5b. “C5 inhibitors,” according to the invention, include any compound or composition that inhibits the processing or cleavage of the pre-cleaved complement component C5 complex or the cleavage products of the complement component C5.

It is understood that inhibition of C5 cleavage prevents the assembly and activity of the cytolytic membrane attack complex (MAC) on glycosylphosphatidylinositol (GPI) adherent protein-deficient erythrocytes. As such, in some cases, C5 inhibitors of the invention may also bind C5b, preventing C6 binding and subsequent assembly of the C5b-9 MAC.

In some embodiments, therapeutic agents include C5 inhibitors. Such C5 inhibitors may include any those presented in Table 1 of US Publication No. US20170137468, the content of which is herein incorporated by reference in its entirety. Therapeutic agents may include R5000 as presented in International Publication No. WO2017/105939. R5000 is a C5 inhibitor with a core amino acid sequence of [cyclo(1,6)]Ac—K-V-E-R-F-D-(N-Me)D-Tbg-Y-azaTrp-E-Y—P-Chg-K (SEQ ID NO: 1). R5000 includes 15 amino acids (all L-amino acids), including 4 unnatural amino acids [N-methyl-aspartic acid or “(N-Me)D”, tert-butylglycine or “Thg”, 7-azatryptophan or “azaTrp”, and cyclohexylglycine or “Chg” ]; a lactam bridge between K1 and D6 of the polypeptide sequence; and a C-terminal lysine reside with a modified side chain, forming a N-ε-(PEG24-γ-glutamic acid-N-α-hexadecanoyl)lysine residue:

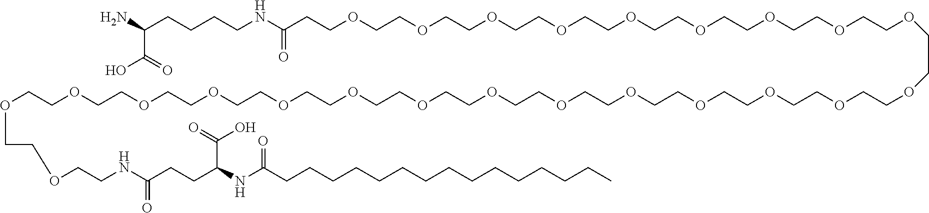 [SMILES: CCCCCCCCCCCCCCCC(=O)N[C@@H](CCC(=O)NCCOCCOCCOCCOCCOCCOCCOCCOCCOCCOCCOCCOCCOCCOCCOCCOCCOCCOCCOCCOCCOCCOCCOCCOCCC(=O)NCCCC[C@H](N)C(=O)O)C(=O)O]

The side chain modification of the C-terminal lysine of R5000 includes a polyethyleneglycol (PEG) spacer, PEG24, with the PEG24 being attached to an L-γ glutamic acid residue that is derivatized with a palmitoyl group.

### Formulations

In some embodiments, the present disclosure provides formulations suitable for delivering therapeutic agents to subjects upon administration of the formulations. As used herein, a “formulation” is a combination of components, wherein the components may include, but are not limited to, solid components, liquid components, and combinations thereof.

Formulations of the present disclosure include controlled release formulations. As used herein, a controlled release formulation is a formulation that modulates the diffusion of one or more formulation components to a surrounding environment. Some controlled release formulations control the release of therapeutic agents due to interactions between the therapeutic agents and the surrounding formulation matrix formed by formulation components. Sustained release formulations are controlled release formulations that slow or prolong the diffusion of one or more formulation components. Sustained release formulations may slow the diffusion of therapeutic agents from the formulation to surrounding environments. The ability of therapeutic agents to diffuse from some sustained release formulations may be influenced by formulation viscosity. For injectable formulations, less viscous formulations are desirable to facilitate injection. Conversely, more highly viscous formulations are often more suitable for slowing the diffusion of therapeutic agents from the formulation.

In some embodiments, sustained release formulations of the present disclosure increase in viscosity upon administration. Such formulations may facilitate injection while slowing the diffusion of a therapeutic agent as the formulation viscosity increases after injection. By formulating specific components in carefully selected ratios with a therapeutic agent, sustained release formulations may be generated that form an in-situ depot upon injection with sustained release properties.

Sustained release formulations may include a therapeutic agent and at least one acylglycerol. Some sustained release formulations may include a mixture of acylglycerols. Such mixtures may include one or of monoglycerides, diglycerides, and triglycerides. Sustained release formulations may be low viscosity formulations suitable for parenteral dosing. Sustained release properties are unexpected for this class of formulations (see United States Publication No. US20140162944). The present disclosure provides formulations that rapidly form highly viscous non-lamellar liquid crystalline phases upon absorption of bodily aqueous fluids when the formulations include a combination of monoglycerides and peptide-based therapeutic agents (e.g., R5000).

Some monoglycerides are solid at room temperature, creating issues with high viscosity in formulations prepared for parenteral injection. Diglycerides are relatively low in viscosity and can also generate relatively low viscosity non-lamellar liquid crystalline phases in the subcutaneous or intramuscular space. This has been demonstrated by others with diacylglycerol formulations that include phosphatidyl choline (see United States Publication No. US20140162944, the contents of which are herein incorporated by reference in their entirety). These low viscosity injectable formulations transform into a non-lamellar liquid crystalline structure upon exposure to body fluid. Unlike these previous formulations, embodiments of the present disclosure provide low viscosity formulations suitable for subcutaneous, self-administration by combining therapeutic agents (e.g., R5000) with acylglycerol mixtures that include specific types and ratios of mono-, di-, and tri-glycerides. Some formulations may be prepared without phospholipid (e.g., phosphatidyl choline) components without losing the ability to transform into a non-lamellar liquid crystalline phase upon administration.

In some embodiments, sustained release formulations of the present disclosure may be optimized to maintain therapeutic agent levels in a subject within the therapeutic window. Optimization may be carried out by modulating the concentration of therapeutic agents. Some formulations may be optimized by modulating the type of acylglycerols included. This may include incorporating acylglycerols carrying different lipid chain lengths and/or degrees of unsaturation. Some formulations may be optimized by modulating the ratio of mono-, di-, and tri-glycerides in acylglycerol mixtures. In some embodiments, sustained release formulations may include acylglycerol mixtures that include acylglycerols with different headgroups. Some acylglycerols do not include a head group. Such acylglycerols may include glycerol backbones with only hydroxyl groups at non-lipidated carbons. Some acylglycerols include phosphoric acid head groups. Some acylglycerols include phosphocholine head groups.

Sustained release formulations of the present disclosure may include a therapeutic agent and a mixture of acylglycerols, wherein the mixture includes from about 40% to about 70% monoglycerides; from about 0% to about 60% diglycerides; and from about 0% to about 60% triglycerides. Acylglycerols from the mixture may include one or more of long chain fatty acids and medium chain fatty acids. The acylglycerols may include one or more of saturated fatty acids and unsaturated fatty acids. Long chain fatty acids may include, but are not limited to, linoleic acid and oleic acid. Medium chain fatty acids may include, but are not limited to, capric acid and caprylic acid. The acylglycerol mixture may include about 60% monoglycerides. The monoglycerides may include linoleic acid. The acyglycerol mixture may include about 20% diglycerides. The diglycerides may include capric acid. The acylglycerol mixture may include about 20% triglycerides. The triglycerides may include capric acid.

In some embodiments, sustained release formulations may include an acylglycerol mixture, wherein the acylglycerol mixture includes one or more of PECEOL® (Gattefosse, Saint Priest, France), MAISINE® 35-1 (Gattefosse, Saint Priest, France), MAISINE® CC (Gattefosse, Saint Priest, France), monoolein (MC18-1), monolinolein (MC18-2), dilinolein (DC18-2), trilinolein (TC18-2), monolinolenin (MC18-3), dilinolenin (DC18-3), trilinolenin (TC18-3), dicaprylin (DC8-0), tricaprylin (TC8-0), dicaprin (DC10-0), tricaprin (TC10-0), and diglycerophosphocholine [with C8 (DC8-0PC) or C10 (DC10-0PC) lipid chains]. In some embodiments, sustained release formulations include acylglycerol mixtures with from about 40% to about 70% MC18-2. The mixtures may include about 60% MC18-2. Some sustained release formulations include acylglycerol mixtures with from about 0% to about 50% MC18-1. The mixtures may include about 50% MC18-1. Some sustained release formulations include acylglycerol mixtures with from about 0% to about 50% DC18-2. The mixtures may include about 15% DC18-2. Some sustained release formulations include acylglycerol mixtures with from about 0% to about 40% TC18-2. Some sustained release formulations include acylglycerol mixtures with from about 0% to about 20% DC8-0. The DC8-0 may include phosphocholine (DC8-0PC). Some sustained release formulations include acylglycerol mixtures with from about 0% to about 20% DC10-0. The DC10-0 may include phosphocholine (DC10-0PC). Some mixtures may include about 20% DC10-0. Some sustained release formulations may include acylglycerol mixtures with from about 0% to about 40% TC8-0. Some sustained release formulations may include acylglycerol mixtures with from about 00 to about 60% TC10-0. The mixtures may include about 20% TC10-0. Some sustained release formulations include acylglycerol mixtures with MC18-2, DC10-0, and TC10-0 at a ratio of 60:20:20.

In some embodiments, sustained release formulations may include acylglycerol mixtures according to any of those listed in Table 1. In the Table, “*” indicates inclusion of a phosphocholine head group.

TABLE 1
Acylglycerol mixtures

| | Mix percentage | | | | | | | |
|---|---|---|---|---|---|---|---|---|
| | MC | MC | DC | TC | DC | TC | DC | TC |
| Mix # | 18-1 | 18-2 | 18-2 | 18-2 | 8-0 | 8-0 | 10-0 | 10-0 |
| 1 | | 50 | 15 | 35 | | | | |
| 2 | | 70 | 15 | 15 | | | | |
| 3 | 50 | 50 | | | | | | |
| 4 | | 70 | | | 20 | 10 | | |
| 5 | | 40 | | | 20* | 40 | | |
| 6 | | 70 | | | 5* | 25 | | |
| 7 | | 60 | | | | | 20 | 20 |
| 8 | | 40 | | | | | 5* | 55 |
| 9 | | 50 | | | | | 5* | 45 |
| 10 | | 41 | 47 | 12 | | | | |

Sustained release formulations described herein may further include one or more excipient. In some embodiments, sustained release formulations may include one or more of phosphate buffered saline (PBS), propylene glycol, a surfactant, a co-solvent, and an antioxidant. In some embodiments, co-solvents may include one or more of nonionic surfactants, anionic surfactants, polyethylene glycol, polyethylene glycol 300, and propylene glycol. In some embodiments, surfactants may included one or more of poloxamer 407, TWEEN® 80 (Sigma-Aldrich, St. Louis, Mo.), polysorbate 80, polyoxyethylene (20) sorbitan monooleate, bile salt, and sodium deoxycholate (Na-DC).

In some embodiments, concentrations of therapeutic agents included in formulations may alter the nature of liquid crystal phase formulation properties. Such properties may be determined using small angle X-ray scattering (SAXS) analysis. SAXS uses X-ray scattering to analyze the size, shape, and distribution of formulation particles.

In some embodiments, sustained release formulations include a therapeutic agent that inhibits complement activity. The complement inhibitor may be a C5 inhibitor. The C5 inhibitor may be R5000.

The therapeutic agent (e.g., R5000) may be present in sustained release formulations at concentrations of from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 20 mg/mL, from about 5 mg/mL to about 30 mg/mL, from about 10 mg/mL to about 40 mg/mL, from about 15 mg/mL to about 50 mg/mL, from about 20 mg/mL to about 75 mg/mL, from about 25 mg/mL to about 100 mg/mL, from about 30 mg/mL to about 125 mg/mL, from about 35 mg/mL to about 150 mg/mL, from about 40 mg/mL to about 175 mg/mL, from about 45 mg/mL to about 200 mg/mL, from about 50 mg/mL to about 225 mg/mL, from about 60 mg/mL to about 250 mg/mL, from about 70 mg/mL to about 300 mg/mL, from about 80 mg/mL to about 350 mg/mL, from about 90 mg/mL to about 400 mg/mL, from about 100 mg/mL to about 450 mg/mL, from about 110 mg/mL to about 500 mg/mL, from about 120 mg/mL to about 600 mg/mL, from about 130 mg/mL to about 700 mg/mL, from about 140 mg/mL to about 800 mg/mL, from about 150 mg/mL to about 900 mg/mL, from about 200 mg/mL to about 1000 mg/mL, or more than 1000 mg/ml. In some embodiments, sustained release formulations include R5000 at a concentration of about 130 mg/ml.

Sustained release formulations may be used according to methods of the present disclosure for delivering therapeutic agents to subjects. Such methods may include preparing a sustained release formulation of the present disclosure and administering the formulation to a subject. The formulation may be a low viscosity formulation. The formulation may become highly viscous upon administration to the subject. The formulation may form a highly viscous non-lamellar liquid crystalline phase upon administration to the subject. Transformation of formulations to non-lamellar liquid crystalline phase may occur upon contact between the formulations and an aqueous bodily fluid. Therapeutic agents may be continuously released from formulations over an extended period of time after administration.

### Therapeutic Indications

In some embodiments, the present disclosure provides methods of treating therapeutic indications using compounds and formulations described herein. A “therapeutic indication,” as used herein, refers to any disease, disorder, condition, or symptom that may be alleviated, cured, improved, reversed, stabilized, or otherwise addressed through one or more forms of therapeutic intervention (e.g., therapeutic agent administration or specific treatment method).

Therapeutic indications may include complement-related indications. As used herein, the term “complement-related indication” refers to any disease, disorder, condition, or symptom related to the complement system, e.g., cleavage or processing of a complement component, such as C5. Complement-related indications may include, but are not limited to ocular indications, autoimmune diseases and disorders, neurological diseases and disorders, vascular and blood diseases and disorders, inflammatory indications, wounds and injuries, kidney-related indications, infectious diseases and disorders, and pregnancy-related indications. Experimental evidence suggests that many complement-related indications are alleviated through inhibition of complement activity. In some embodiments, methods of the present disclosure include treating complement-related indications with formulations presented herein.

In some embodiments, methods of the disclosure include treating complement-related indications by inhibiting complement activity in a subject using formulations presented herein. In some cases, the percentage of complement activity inhibited in a subject may be at least 10%, at least 20%, at least 30%, at least 40%, at least 50%, at least 60%, at least 70%, at least 80%, at least, 85%, at least 90%, at least 95%, at least 96%, at least 97%, at least 98%, at least 99%, at least 99.5%, or at least 99.9%. In some cases, this level of inhibition and/or maximum inhibition of complement activity may be achieved by from about 1 hour after an administration to about 3 hours after an administration, from about 2 hours after an administration to about 4 hours after an administration, from about 3 hours after an administration to about 10 hours after an administration, from about 5 hours after an administration to about 20 hour after an administration, or from about 12 hours after an administration to about 24 hours after an administration. Inhibition of complement activity may continue throughout a period of at least 1 day, of at least 2 days, of at least 3 days, of at least 4 days, of at least 5 days, of at least 6 days, of at least 7 days, of at least 2 weeks, of at least 3 weeks, or at least 4 weeks.

In some cases, this level of inhibition may be achieved through daily administration. Such daily administration may include administration for at least 2 days, for at least 3 days, for at least 4 days, for at least 5 days, for at least 6 days, for at least 7 days, for at least 2 weeks, for at least 3 weeks, for at least 4 weeks, for at least 2 months, for at least 4 months, for at least 6 months, for at least 1 year, or for at least 5 years. In some cases, subjects may be administered compounds or compositions of the present disclosure for the life of such subjects.

In some embodiments, compounds and formulations described herein provide an extended therapeutic window from a single administration. Such compounds and formulations include sustained release formulations. Sustained release formulations may be used to treat any of the therapeutic indications (e.g., complement-related indications) described herein.

In some embodiments, the present disclosure provides methods of treating complement-related indications by inhibiting C5 activity in a subject. “C5-dependent complement activity” or “C5 activity,” as used herein refers to activation of the complement cascade through cleavage of C5, the assembly of downstream cleavage products of C5, or any other process or event attendant to, or resulting from, the cleavage of C5. In some cases, the percentage of C5 activity inhibited in a subject may be at least 10%, at least 20%, at least 30%, at least 40%, at least 50%, at least 60%, at least 70, at least 80%, at least, 85%, at least 90%, at least 95%, at least 96%, at least 97%, at least 98%, at least 99%, at least 99.5%, or at least 99.9%.

As used herein the terms “treat,” “treatment,” and the like, refer to relief from or alleviation of pathological processes. In the context of the present invention insofar as it relates to any of the other conditions recited herein below, the terms “treat,” “treatment,” and the like mean to relieve or alleviate at least one symptom associated with such condition, or to slow or reverse the progression or anticipated progression of such condition, such as slowing reducing the destruction of red blood cells (as measured by reduced transfusion requirements or increased hematocrit or hemoglobin levels) resulting from paroxysmal nocturnal hemoglobinuria.

By “lower” or “reduce” in the context of a disease marker or symptom is meant a statistically significant decrease in such level. The decrease can be, for example, at least 10%, at least 20%, at least 30%, at least 40% or more, and is preferably down to a level accepted as within the range of normal for an individual without such disorder.

By “increase” or “raise” in the context of a disease marker or symptom is meant a statistically significant rise in such level. The increase can be, for example, at least 10%, at least 20%, at least 30%, at least 40% or more, and is preferably up to a level accepted as within the range of normal for an individual without such disorder.

As used herein, the phrases “therapeutically effective amount” and “prophylactically effective amount” refer to an amount that provides a therapeutic benefit in the treatment, prevention, or management of pathological processes or an overt symptom of one or more pathological processes. The specific amount that is therapeutically effective can be readily determined by an ordinary medical practitioner and may vary depending on factors known in the art, such as, for example, the type of pathological processes, patient history and age, the stage of pathological processes, and the administration of other agents that inhibit pathological processes.

As used herein, a “pharmaceutical composition” comprises a pharmacologically effective amount of a compound and a pharmaceutically acceptable carrier. As used herein, “pharmacologically effective amount,” “therapeutically effective amount” or simply “effective amount” refers to that amount of a compound effective to produce the intended pharmacological, therapeutic or preventive result. For example, if a given clinical treatment is considered effective when there is at least a 10% alteration (increase or decrease) in a measurable parameter associated with a disease or disorder, a therapeutically effective amount of a drug for the treatment of that disease or disorder is the amount necessary to affect at least a 10% alteration in that parameter. For example, a therapeutically effective amount of a compound may be one that alters binding of a target to its natural binding partner by at least 10%.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of a compound or pharmaceutical composition thereof, “effective against” a disease or disorder indicates that administration in a clinically appropriate manner results in a beneficial effect for at least a fraction of patients, such as an improvement of symptoms, a cure, a reduction in disease load, reduction in tumor mass or cell numbers, extension of life, improvement in quality of life, a reduction in the need for blood transfusions or other effect generally recognized as positive by medical doctors familiar with treating the particular type of disease or disorder.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given compound drug or formulation of that drug can also be judged using an experimental animal model for the given disease as known in the art. When using an experimental animal model, efficacy of treatment is evidenced when a statistically significant modulation in a marker or symptom is observed.

### Paroxysmal Nocturnal Hemoglobinuria (PNH)

In some embodiments, C5 inhibitor compounds (e.g., R5000) and sustained release formulations thereof may be used to treat, prevent or delay development of paroxysmal nocturnal hemoglobinuria (PNH). In some embodiments, the treatment may be involved with the prevention of hemolysis of PNH erythrocytes in a dose dependent manner.

An acquired mutation in the phosphatidylinositol glycan anchor biosynthesis, class A (PIG-A) gene that originates from a multipotent hematopoietic stem cell results in a rare disease known as paroxysmal nocturnal hemoglobinuria (PNH) (Pu, J. J. et al., Paroxysmal nocturnal hemoglobinuria from bench to bedside. Clin Transl Sci. 2011 June; 4(3):219-24). PNH is characterized by bone marrow disorder, hemolytic anemia and thrombosis. The PIG-A gene product is necessary for the production of a glycolipid anchor, glycosylphosphatidylinositol (GPI), utilized to tether proteins to the plasma membrane. Two complement-regulatory proteins, CD55 and CD59, become nonfunctional in the absence of GPI. This leads to complement-mediated destruction of these cells. C5 inhibitors are particularly useful in the treatment of PNH. In some embodiments, sustained release formulations of C5 inhibitors may be used to treat, prevent or delay development of Paroxysmal nocturnal hemoglobinuria (PNH) or anemias associated with complement. Subjects with PNH are unable to synthesize functional versions of the complement regulatory proteins CD55 and CD59 on hematopoietic stem cells. This results in complement-mediated hemolysis and a variety of downstream complications. As used herein, the term “downstream” or “downstream complication” refers to any event occurring after and as a result of another event. In some cases, downstream events are events occurring after and as a result of C5 cleavage and/or complement activation.

PNH is characterized by low hemoglobin, increased levels of lactate dehydrogenase and bilirubin, and decreased level of haptoglobin. Symptoms of PNH include symptoms of anemia, such as tiredness, headaches, dyspnea, chest pain, dizziness, and feeling of lightheadedness.

Current treatments for PNH include the use of eculizumab (Alexion Pharmaceuticals, Cheshire, Conn.). In some cases, eculizumab may be ineffective due to mutation in C5, short half-life, immune reaction, or other reason. In some embodiments, methods of the present disclosure include methods of treating subjects with PNH, wherein such subjects have been treated previously with eculizumab. In some cases, eculizumab is ineffective in such subject, making treatment with compounds of the present disclosure important for therapeutic relief. In some embodiments, compounds of the present disclosure may be used to treat subjects that are resistant to eculizumab treatment. Such subjects may include subjects with the R885H/C polymorphism, which confers resistance to eculizumab. In some cases, compounds of the present disclosure are administered simultaneously or in conjunction with eculizumab therapy. In such cases, subjects may experience one or more beneficial effects of such combined treatment, including, but not limited to more effective relief, faster relief and/or fewer side effects.

### Inflammatory Indications

Complement-related indications may include inflammatory indications. As used herein, the term “inflammatory indication” refers to therapeutic indications that involve immune system activation. Inflammation may be upregulated during the proteolytic cascade of the complement system. Although inflammation may have beneficial effects, excess inflammation may lead to a variety of pathologies (Markiewski et al. 2007. Am J Pathol. 17: 715-27). In some embodiments, C5 inhibitor compounds and sustained release formulations thereof may be used to treat complement-related indications that include inflammatory indications.

Inflammatory indications may include, but are not limited to, Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Acute antibody-mediated rejection following organ transplantation, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Autoimmune urticaria, Axonal & neuronal neuropathies, Bacterial sepsis and septic shock, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal osteomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Diabetes Type I, Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia (including atypical hemolytic uremic syndrome and plasma therapy-resistant atypical hemolytic-uremic syndrome), Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Insulin-dependent diabetes (typel), Interstitial cystitis, Juvenile arthritis, Juvenile diabetes, Kawasaki syndrome, Lambert-Eaton syndrome, Large vessel vasculopathy, Leukocytoclastic vasculitis, Lichen planus, Lichen sclerosus, Ligneous conjunctivitis, Linear IgA disease (LAD), Lupus (SLE), Lyme disease, Meniere's disease, Microscopic polyangiitis, Mixed connective tissue disease (MCTD), Mooren's ulcer, Mucha-Habermann disease, Multiple endocrine neoplasia syndromes, Multiple sclerosis, Multifocal motor neuropathy, Myositis, Myasthenia gravis, Narcolepsy, Neuromyelitis optica (Devic's), Neutropenia, Ocular cicatricial pemphigoid, Optic neuritis, Osteoarthritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polyendocrinopathies, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic Pulmonary fibrosis, Pyoderma gangrenosum, Pure red cell aplasia, Raynauds phenomenon, Reactive arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Shiga-Toxin producing Escherichia Coli Hemolytic-Uremic Syndrome (STEC-HUS), Sjogren's syndrome, Small vessel vasculopathy, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Tubular autoimmune disorder, Ulcerative colitis, Undifferentiated connective tissue disease (UCTD), Uveitis, Vesiculobullous dermatosis, Vasculitis, Vitiligo, and Wegener's granulomatosis (also known as Granulomatosis with Polyangiitis (GPA)).

### Sterile Inflammation

Inflammatory indications may include sterile inflammation. Sterile inflammation is inflammation that occurs in response to stimuli other than infection. Sterile inflammation may be a common response to stress such as genomic stress, hypoxic stress, nutrient stress or endoplasmic reticulum stress caused by a physical, chemical, or metabolic noxious stimuli. Sterile inflammation may contribute to pathogenesis of many diseases such as, but not limited to, ischemia-induced injuries, rheumatoid arthritis, acute lung injuries, drug-induced liver injuries, inflammatory bowel diseases and/or other diseases, disorders or conditions. Mechanism of sterile inflammation and methods and compounds for treatment, prevention and/or delaying of symptoms of sterile inflammation may include any of those taught by Rubartelli et al. in Frontiers in Immunology, 2013, 4:398-99, Rock et al. in Annu Rev Immunol. 2010, 28:321-342 or in U.S. Pat. No. 8,101,586, the contents of each of which are herein incorporated by reference in their entirety. In some embodiments, C5 inhibitor compounds and sustained release formulations thereof may be used to treat, prevent or delay development of sterile inflammation.

### Systemic Inflammatory Response (SIRS) and Sepsis

Inflammatory indications may include systemic inflammatory response syndrome (SIRS). SIRS is inflammation affecting the whole body. Where SIRS is caused by an infection, it is referred to as sepsis. SIRS may also be caused by non-infectious events such as trauma, injury, burns, ischemia, hemorrhage and/or other conditions. During sepsis and SIRS, complement activation leads to excessive generation of complement activation products which may cause multi organ failure (MOF) in subjects. In some embodiments, C5 inhibitor compounds and sustained release formulations thereof, may be used to treat and/or prevent SIRS. C5 inhibitor formulations may be used to control and/or balance complement activation for prevention and treatment of SIRS, sepsis and/or MOF. The methods of applying complement inhibitors to treat SIRS and sepsis may include those taught by Rittirsch et al. in Clin Dev Immunol, 2012, 962927, in U.S. publication No. US2013/0053302 or in U.S. Pat. No. 8,329,169, the contents of each of which are herein incorporated by reference in their entirety.

### Acute Respiratory Distress Syndrome (ARDS)

Inflammatory indications may include acute respiratory distress syndrome (ARDS). ARDS is a widespread inflammation of the lungs and may be caused by trauma, infection (e.g., sepsis), severe pneumonia and/or inhalation of harmful substances. ARDS is typically a severe, life-threatening complication. Studies suggest that neutrophils may contribute to development of ARDS by affecting the accumulation of polymorphonuclear cells in the injured pulmonary alveoli and interstitial tissue of the lungs. In some embodiments, C5 inhibitor formulations of the present disclosure may be used to treat and/or prevent development of ARDS. C5 inhibitor formulations may be administered to reduce and/or prevent tissue factor production in alveolar neutrophils. C5 inhibitor formulations may further be used for treatment, prevention and/or delaying of ARDS, in some cases according to any of the methods taught in International publication No. WO2009/014633, the contents of which are herein incorporated by reference in their entirety.

### Periodontitis

Inflammatory indications may include periodontitis. Periodontitis is a widespread, chronic inflammation leading to the destruction of periodontal tissue which is the tissue supporting and surrounding the teeth. The condition also involves alveolar bone loss (bone that holds the teeth). Periodontitis may be caused by a lack of oral hygiene leading to accumulation of bacteria at the gum line, also known as dental plaque. Certain health conditions such as diabetes or malnutrition and/or habits such as smoking may increase the risk of periodontitis. Periodontitis may increase the risk of stroke, myocardial infarction, atherosclerosis, diabetes, osteoporosis, pre-term labor, as well as other health issues. Studies demonstrate a correlation between periodontitis and local complement activity. Periodontal bacteria may either inhibit or activate certain components of the complement cascade. In some embodiments, C5 inhibitor formulations of the present disclosure may be used to treat or prevent development of periodontitis and/or associated conditions. Complement activation inhibitors and treatment methods may include any of those taught by Hajishengallis in Biochem Pharmacol. 2010, 15, 80(12): 1 and Lambris or in US publication No. US2013/0344082, the contents of each of which are herein incorporated by reference in their entirety.

### Rheumatoid Arthritis

Inflammatory indications may include rheumatoid arthritis. Rheumatoid arthritis is an autoimmune condition affecting the wrists and small joints of the hands. Typical symptoms include pain, stiffness of the joints, swelling, and feeling of warmth. Activated components of the complement system affect development of rheumatoid arthritis, as products of complement cascade mediate proinflammatory activities, such as vascular permeability and tone, leukocyte chemotaxis and the activation and lysis of multiple cell types (see Wang, et al., Proc. Natl. Acad. Sci., 1995; 92: 8955-8959). Wang et al. demonstrated that inhibition of C5 complement cascade in animals prevented the onset of arthritis and ameliorated established condition. Complement activation inhibitors and treatment methods may include any of those taught by Wang, et al., Proc. Natl. Acad. Sci., 1995; 92: 8955-8959, the contents of which are herein incorporated by reference in their entirety. In some embodiments, C5 inhibitor formulations of the present disclosure may be used to treat or prevent development of rheumatoid arthritis.

### Asthma

Inflammatory indications may include asthma. Asthma is a chronic inflammation of the bronchial tubes, which are the airways allowing air to pass in and out of the lungs. The condition is characterized by narrowing, inflammation and hyperresponsiveness of the tubes. Typical symptoms include periods of wheezing, chest tightness, coughing and shortness of breath. Asthma the most common respiratory disorder. Complement proteins C3 and C5 are associated with many pathophysiological features of asthma, such as inflammatory cell infiltration, mucus secretion, increased vascular permeability, and smooth muscle cell contraction, and therefore it has been suggested that downregulation of complement activation may be used to treat, manage or prevent asthma. In some embodiments, C5 inhibitor formulations of the present disclosure may be used to treat or prevent development of asthma. Complement activation inhibitors and treatment methods may include any of those taught by Khan et al., Respir Med. 2014 April; 108(4): 543-549, the contents of which are herein incorporated by reference in their entirety.

### Anaphylaxis

Inflammatory indications may include anaphylaxis. Anaphylaxis is a severe and potentially life-threatening allergic reaction. Anaphylaxis may lead to a shock characterized e.g. by sudden drop of blood pressure, narrowing of airways, breathing difficulties, rapid and weak pulse, a rash, nausea and vomiting. The cardiopulmonary collapse during anaphylaxis has been associated with complement activation and generation of C3a and C5a anaphylatoxins. Balzo et al. report animal studies indicating that complement activation markedly enhance cardiac dysfunction during anaphylaxis (Balzo et al., Circ Res. 1989 September; 65(3):847-57). Complement activation inhibitors and treatment methods may include any of those taught by Balzo et al., the contents of which are herein incorporated by reference in their entirety. In some embodiments, C5 inhibitor formulations of the present disclosure may be used to treat or prevent development of anaphylaxis.

### Bowel Inflammation

Inflammatory indications may include inflammatory bowel disease (IBD). IBD is a reoccurring condition with periods of mild to severe inflammation or periods of remission. Common symptoms include diarrhea, fatigue and fever, abdominal pain, weight loss, reduced appetite and bloody stool. Types of IBD include ulcerative proctitis, dextran sulfate sodium colitis, proctosigmoitidis, left-sided colitis, panconlitis, acute severe ulcerative colitis. IBD, such as dextran sulfate sodium colitis and ulcerative colitis, have been associated with C5 complement (Webb et al., Int J Med Pharm Case Reports. 2015, 4(5): 105-112 and Aomatsu et al., J Clin Biochem Nutr. 2013; 52(1):72-5). Complement activation inhibitors and treatment methods may include any of those taught by Webb et al. or Aomatsu et al, the contents of each of which are herein incorporated by reference in their entirety. In some embodiments, C5 inhibitor formulations of the present disclosure may be used to treat or prevent development of IBD.

### Systemic Inflammation During Cardiopulmonary Bypass

Inflammatory indications may include inflammatory response induced by cardiopulmonary bypass (CBP). CBP is a technique used during surgery to take over the function of heart and lungs to maintain blood circulation and oxygen concentration of the blood. CBD provokes a systemic inflammatory response that may lead to complications of the surgical patients. The suggested cause may be due to contact activation of blood with artificial surfaces during extracorporeal circulation. The inflammation response may lead to SIRS and be life-threatening.

Complement activation has been associated with the inflammatory response induced by CBP. Studies have suggested that terminal components C5a and C5b-9 directly contribute to platelet and neutrophil activation during the extracorporeal blood circulation and C5 has been identified as a therapeutic site for prevention and treatment of inflammatory response induced by CBP (Rinder et al. J Clin Invest. 1995; 96(3): 1564-1572). Complement activation inhibitors and treatment methods may include any of those taught by Rinder et al. J Clin Invest. 1995; 96(3): 1564-1572, the contents of which are herein incorporated by reference in their entirety. In some embodiments, C5 inhibitor formulations of the present disclosure may be used to treat or prevent development of inflammatory response induced by CBP.

### Rejection in Organ or Tissue Transplant

Inflammatory indications may include immune rejection of transplants. Transplants may be organs (e.g. heart, kidneys, liver, lungs, intestine, thymus and pancreas) or tissues (e.g. bones, tendons, skin, cornea, veins). Different types of transplants include autograft (transplanting patient's own tissue), allograft (transplant between two members of the same species) or xenograft (transplant between members of different species, e.g. from an animal to a human). Complications after organ transplant arise as the recipient's immune system attacks the transplanted tissue. The rejection may be hyperacute referring to a reaction occurring within few minutes after the transplant is performed, and typically occurs when the antigens are unmatched. Acute rejection occurs within a week or few months after transplant. Some rejections are chronic and take place over many years.

Transplant rejection and related inflammation has been associated with complement system. The complement cascade is relevant to transplantation in a number of ways, e.g. as an effector mechanism of antibody-initiated allograft injury, promotion of ischemia-reperfusion injury, and formation and function of alloantibodies (Sheen and Heeger, Curr Opin Organ Transplant. 2015; 20(4):468-75). Therapy targeting complement has been suggested to have significance for the survival and health of transplant patients As an example, studies have shown that C5 blockage of C5 with eculizumab reduces the incidence of early antibody-mediated rejection (AMR) of organ allografts (Stegall et al., Nature Reviews Nephrology 8(11):670-8, 2012) and inhibition of C5 may prevent acute cardiac tissue injury in an ex vivo model of pig-to-human xenotransplantation (Kroshus et al, Transplantation. 1995, 15; 60(11): 1194-202.) Complement activation inhibitors and treatment methods may include any of those taught by Stegall et al., Nature Reviews Nephrology 8(11):670-8, 2012 and Kroshus et al, Transplantation. 1995, 15; 60(11):1194-202, and (Sheen and Heeger, Curr Opin Organ Transplant. 2015; 20(4):468-75, the contents of each of which are herein incorporated by reference in their entirety. In some embodiments, C5 inhibitor formulations of the present disclosure may be used to treat subjects with or receiving transplanted organs or tissues.

### Wounds and Injuries

Complement-related indications may include wounds and injuries. As used herein, the term “injury” typically refers to physical trauma, but may include localized infection or disease processes. Injuries may be characterized by harm, damage or destruction caused by external events affecting body parts and/or organs. Non-limiting examples of injuries include head trauma and crush injuries. Wounds are associated with cuts, blows, burns and/or other impacts to the skin, leaving the skin broken or damaged. Wounds and injuries are often acute but if not healed properly they may lead to chronic complications and/or inflammation. In some embodiments, C5 inhibitor formulations of the present disclosure may be used to treat and/or promote healing of different types of wounds and/or injuries.

### Wounds and Burn Wounds

In some embodiments, C5 inhibitor formulations of the present disclosure may be used to treat and/or to promote healing of wounds. Healthy skin provides a waterproof, protective barrier against pathogens and other environmental effectors. The skin also controls body temperature and fluid evaporation. When skin is wounded these functions are disrupted making skin healing challenging. Wounding initiates a set of physiological processes related to the immune system that repair and regenerate tissue. Complement activation is one of these processes. Complement activation studies have identified several complement components involved with wound healing as taught by van de Goot et al. in J Burn Care Res 2009, 30:274-280 and Cazander et al. Clin Dev Immunol, 2012, 2012:534291, the contents of each of which are herein incorporated by reference in their entirety. In some cases, complement activation may be excessive, causing cell death and enhanced inflammation (leading to impaired wound healing and chronic wounds). In some cases, C5 inhibitor formulations may be used to reduce or eliminate such complement activation to promote wound healing. Treatment with C5 inhibitor formulations may be carried out according to any of the methods for treating wounds disclosed in International Publication No. WO2012/174055, the contents of which are herein incorporated by reference in their entirety.

### Head Trauma

In some embodiments, C5 inhibitor formulations of the present disclosure may be used to treat and/or promote healing of head trauma. Head traumas include injuries to the scalp, the skull or the brain. Examples of head trauma include, but are not limited to concussions, contusions, skull fracture, traumatic brain injuries and/or other injuries. Head traumas may be minor or severe. In some cases, head trauma may lead to long term physical and/or mental complications or death. Studies indicate that head traumas may induce improper intracranial complement cascade activation, which may lead to local inflammatory responses contributing to secondary brain damage by development of brain edema and/or neuronal death (Stahel et al. in Brain Research Reviews, 1998, 27: 243-56, the contents of which are herein incorporated by reference in their entirety). In some embodiments, C5 inhibitor formulations may be used to reduce or prevent related secondary complications of head trauma. Methods of using C5 inhibitor formulations to control complement cascade activation in head trauma may include any of those taught by Holers et al. in U.S. Pat. No. 8,911,733, the contents of which are herein incorporated by reference in their entirety.

### Crush Injury

In some embodiments, C5 inhibitor formulations of the present disclosure may be used to treat and/or promote healing of crush injuries. Crush injuries are injuries caused by a force or a pressure put on the body causing bleeding, bruising, fractures, nerve injuries, wounds and/or other damages to the body. C5 inhibitor formulations may be used to reduce complement activation following crush injuries, thereby promoting healing after crush injuries (e.g. by promoting nerve regeneration, promoting fracture healing, preventing or treating inflammation, and/or other related complications). C5 inhibitor formulations may be used to promote healing according to any of the methods taught in U.S. Pat. No. 8,703,136; International Publication Nos. WO2012/162215; WO2012/174055; or US publication No. US2006/0270590, the contents of each of which are herein incorporated by reference in their entirety.

### Autoimmune Indications

Complement-related indications may include autoimmune indications. The immune system may be divided into innate and adaptive systems, referring to nonspecific immediate defense mechanisms and more complex antigen-specific systems, respectively. The complement system is part of the innate immune system, recognizing and eliminating pathogens. Additionally, complement proteins may modulate adaptive immunity, connecting innate and adaptive responses. Autoimmune diseases and disorders are immune abnormalities causing the system to target self tissues and substances. Autoimmune disease may involve certain tissues or organs of the body. C5 inhibitor formulations of the present disclosure may be used to modulate complement in the treatment and/or prevention of autoimmune diseases. In some cases, such formulations may be used according to the methods presented in Ballanti et al. Immunol Res (2013) 56:477-491, the contents of which are herein incorporated by reference in their entirety.

### Anti-Phospholipid Syndrome (APS) and Catastrophic Anti-Phospholipid Syndrome (CAPS)

Autoimmune indications may include anti-phospholipid syndrome (APS). APS is an autoimmune condition caused by anti-phospholipid antibodies that cause the blood to clot. APS may lead to recurrent venous or arterial thrombosis in organs, and complications in placental circulations causing pregnancy-related complications such as miscarriage, still birth, preeclampsia, premature birth and/or other complications. Catastrophic anti-phospholipid syndrome (CAPS) is an extreme and acute version of a similar condition leading to occlusion of veins in several organs simultaneously. Studies suggest that complement activation may contribute to APS-related complications including pregnancy-related complications, thrombotic (clotting) complications, and vascular complications. In some embodiments, C5 inhibitor formulations of the present disclosure may be used to prevent and/or treat APS by complement activation control. In some cases, C5 inhibitor formulations may be used to treat APS and/or APS-related complications according to the methods taught by Salmon et al. Ann Rheum Dis 2002; 61(Suppl II):ii46-ii50 and Mackworth-Young in Clin Exp Immunol 2004, 136:393-401, the contents of which are herein incorporated by reference in their entirety.

### Cold Agglutinin Disease

Autoimmune indications may include cold agglutinin disease (CAD), also referred to as cold agglutinin-mediated hemolysis. CAD is an autoimmune disease resulting from a high concentration of IgM antibodies interacting with red blood cells at low range body temperatures (Engelhardt et al. Blood, 2002, 100(5): 1922-23). CAD may lead to conditions such as anemia, fatigue, dyspnea, hemoglobinuria and/or acrocyanosis. CAD is related to robust complement activation and studies have shown that CAD may be treated with complement inhibitor therapies. In some embodiments, C5 inhibitor formulations of the present disclosure may be used to treat CAD by inhibiting complement activity. In some cases, C5 inhibitor formulations may be used to treat CAD according to the methods taught by Roth et al in Blood, 2009, 113:3885-86 or in International publication No. WO2012/139081, the contents of each of which are herein incorporated by reference in their entirety.

Dermatological Diseases

Autoimmune indications may include dermatological disease. Skin has a role in a spectrum of immunological reactions and are associated with abnormal or overactivated complement protein functions. Autoimmune mechanisms with autoantibodies and cytotoxic functions of the complement affect epidermal or vascular cells causing tissue damage and skin inflammation (Palenius and Meri, Front Med (Lausanne). 2015; 2: 3). Dermatological diseases associated with autoimmune and complement abnormality include, but are not limited to, hereditary and acquired angioedema, autoimmune urticarial (hives), systemic lupus erythematosus, vasculitis syndromes and urticarial vasculitis, bullous skin diseases (e.g. pemphigus, bullous pemphigioid, mucous membrane pemphigoid, epidermolysis bullosa acquisita, dermatitis herpetiformis, pemphigoides festationis), and partial lipodustrophy. In some cases, C5 inhibitor formulations may be used to treat autoimmune dermatological diseases according to the methods taught by Palenius and Meri, Front Med (Lausanne). 2015; 2: 3, the contents of which are herein incorporated by reference in their entirety. In some embodiments, C5 inhibitor formulations of the present disclosure may be used to treat dermatological diseases.

### Pulmonary Indications

Complement-related indications may include pulmonary indications. Pulmonary indications are therapeutic indications related to the lungs and related airways. Pulmonary indications may include, but are not limited to, asthma, pulmonary fibrosis, chronic obstructive pulmonary disease (COPD), and acute respiratory distress syndrome. In some embodiments, C5 inhibitor formulations of the present disclosure may be used to treat pulmonary indications.

### Cardiovascular Indications

Complement-related indications may include cardiovascular indications. Cardiovascular indications are therapeutic indications related to the heart (cardiac indications) or vasculature (vascular indications). Cardiovascular indications may include, but are not limited to, atherosclerosis, myocardial infarction, stroke, vasculitis, trauma and conditions arising from cardiovascular intervention (including, but not limited to cardiac bypass surgery, arterial grafting and angioplasty). In some embodiments, C5 inhibitor formulations of the present disclosure may be used to treat cardiovascular indications.

Vascular indications are cardiovascular indications related to blood vessels (e.g., arteries, veins, and capillaries). Such indications may affect blood circulation, blood pressure, blood flow, organ function, and/or other bodily functions. In some embodiments, C5 inhibitor formulations of the present disclosure may be used to treat vascular indications.

### Thrombotic Microangiopathy (TMA)

Vascular indications may include thrombotic microangiopathy (TMA) and associated diseases. Microangiopathies affect small blood vessels (capillaries) of the body causing capillary walls to become thick, weak, and prone to bleeding and slow blood circulation. TMAs tend to lead to the development of vascular thrombi, endothelial cell damage, thrombocytopenia, and hemolysis. Organs such as the brain, kidney, muscles, gastrointestinal system, skin, and lungs may be affected. TMAs may arise from medical operations and/or conditions that include, but are not limited to, hematopoietic stem cell transplantation (HSCT), renal disorders, diabetes and/or other conditions. TMAs may be caused by underlying complement system dysfunction, as described by Meri et al. in European Journal of Internal Medicine, 2013, 24: 496-502, the contents of which are herein incorporated by reference in their entirety. Generally, TMAs may result from increased levels of certain complement components leading to thrombosis. In some cases, this may be caused by mutations in complement proteins or related enzymes. Resulting complement dysfunction may lead to complement targeting of endothelial cells and platelets leading to increased thrombosis. In some embodiments, TMAs may be prevented and/or treated with C5 inhibitor formulations of the present disclosure. In some cases, methods of treating TMAs with C5 inhibitor formulations may be carried out according to those described in US publication Nos. US2012/0225056 or US2013/0246083, the contents of each of which are herein incorporated by reference in their entirety.

### Disseminated Intravascular Coagulation (DIC)

Vascular indications may include disseminated intravascular coagulation (DIC). DIC is a pathological condition where the clotting cascade in blood is widely activated and results in formation of blood clots especially in the capillaries. DIC may lead to an obstructed blood flow of tissues and may eventually damage organs. Additionally, DIC affects the normal process of blood clotting that may lead to severe bleeding. C5 inhibitor formulations of the present disclosure may be used to treat, prevent or reduce the severity of DIC by modulating complement activity. In some cases, C5 inhibitor formulations may be used according to any of the methods of DIC treatment taught in U.S. Pat. No. 8,652,477, the contents of which are herein incorporated by reference in their entirety.

### Vasculitis

Vascular indications may include vasculitis. Generally, vasculitis is a disorder related to inflammation of blood vessels, including veins and arteries, characterized by white blood cells attacking tissues and causing swelling of the blood vessels. Vasculitis may be associated with an infection, such as in Rocky Mountain spotted fever, or autoimmunity. An example of autoimmunity associated vasculitis is Anti-Neutrophil Cytoplasmic Autoantibody (ANCA) vasculitis. ANCA vasculitis is caused by abnormal antibodies attacking the body's own cells and tissues. ANCAs attack the cytoplasm of certain white blood cells and neutrophils, causing them to attack the walls of the vessels in certain organs and tissues of the body. ANCA vasculitis may affect skin, lungs, eyes and/or kidney. Studies suggest that ANCA disease activates an alternative complement pathway and generates certain complement components that create an inflammation amplification loop resulting in a vascular injury (Jennette et al. 2013, Semin Nephrol. 33(6): 557-64, the contents of which are herein incorporated by reference in their entirety). In some embodiments, C5 inhibitor formulations of the present disclosure may be used to prevent and/or treat vasculitis. In some cases, C5 inhibitor formulations may be used to prevent and/or treat ANCA vasculitis by inhibiting complement activation.

### Neurological Indications

Complement-related indications may include neurological indications. Neurological indications are therapeutic indications related to the nervous system. Neurological indications may include neurodegeneration. Neurodegeneration generally relates to a loss of structure or function of neurons, including death of neurons. The C5 inhibitor formulations of the present disclosure may be used to prevent, treat and/or ease the symptoms of neurological indications, including, but not limited to neurodegenerative diseases and related disorders. Treatment may include inhibiting the effect of complement on neuronal cells using formulations of the present disclosure. Neurodegenerative related disorders include, but are not limited to, Amyelotrophic Lateral Sclerosis (ALS), Multiple Sclerosis (MS), Parkinson's disease, Alzheimer's disease, and Lewy body dementia.

### Amyotrophic Lateral Sclerosis (ALS)

Neurological indications may include ALS. ALS is a fatal motor neuron disease characterized by the degeneration of spinal cord neurons, brainstems and motor cortex. ALS causes loss of muscle strength leading eventually to a respiratory failure. Complement dysfunction may contribute to ALS, and therefore ALS may be prevented, treated and/or the symptoms may be reduced by therapy with C5 inhibitor formulations targeting complement activity. In some cases, C5 inhibitor formulations of the present disclosure may be used to treat ALS and/or promote nerve regeneration. In some cases, C5 inhibitor formulations may be used as complement inhibitors according to any of the methods taught in US publication No. US2014/0234275 or US2010/0143344, the contents of each of which are herein incorporated by reference in their entirety.

### Alzheimer's Disease

Neurological indications may include Alzheimer's disease. Alzheimer's disease is a chronic neurodegenerative disease with symptoms that may include disorientation, memory loss, mood swings, behavioral problems and eventually loss of bodily functions. Alzheimer's disease is thought to be caused by extracellular brain deposits of amyloid that are associated with inflammation-related proteins such as complement proteins (Sjoberg et al. 2009. Trends in Immunology. 30(2): 83-90, the contents of which are herein incorporated by reference in their entirety). In some embodiments, C5 inhibitor formulations of the present disclosure may be used to prevent and/or treat Alzheimer's disease by controlling complement activity. In some cases, C5 inhibitor formulations may be used according to any of the Alzheimer's treatment methods taught in US publication No. US2014/0234275, the contents of which are herein incorporated by reference in their entirety.

### Multiple Sclerosis and Neuromyelitis Optica

Neurological indications may include multiple sclerosis (MS) or neuromyelitis optica (NMO). MS is an inflammatory condition affecting the central nervous system as the immune system launches an attack against the body's own tissues, and in particular against nerve-insulating myelin. The condition may be triggered by an unknown environmental agent, such as a virus. MS is progressive and eventually results in disruption of the communication between the brain and other parts of the body. Typical early symptoms include blurred vision, partial blindness, muscle weakness, difficulties in coordination and balance, impaired movement, pain and speech impediments. NMO (also known as Devic's disease) is an inflammatory demyelinating disease affecting the optic nerves and spinal cord as the immune system attacks the astrocytes. NMO is sometimes considered as a variant of MS. Typical symptoms of NMO include muscle weakness of the legs or paralysis, loss of senses (e.g. blindness) and dysfunctions of the bladder and bowel.

MS and NMO have been associated with complement component regulation e.g. by pathological and animal model studies (Ingram et al., Clin Exp Immunol. 2009 February; 155(2): 128-139). In the central nervous system glial cells and neurons produce the majority of complement proteins and the expression is increased in response to inflammation. In some embodiments, C5 inhibitor formulations of the present disclosure may be used to treat and/or prevent MS or NMO. Complement activation inhibitors and treatment methods may include any of those taught by Ingram et al., Clin Exp Immunol. 2009 February; 155(2): 128-139, the contents of which are herein incorporated by reference in their entirety.

### Kidney-Related Indications

Complement-related indications may include kidney-related indications. Kidney-related indications are therapeutic indications related to kidneys. Kidneys are organs responsible for removing metabolic waste products from the blood stream. Kidneys regulate blood pressure, the urinary system, and homeostatic functions and are therefore essential for a variety of bodily functions. Kidneys may be more seriously affected by inflammation (as compared to other organs) due to unique structural features and exposure to blood. Kidneys also produce their own complement proteins which may be activated upon infection, kidney disease, and renal transplantations. C5 inhibitor formulations of the present disclosure may be used to treat kidney-related indications, in some cases by inhibiting complement activity. In some cases, C5 inhibitor formulations may be used as complement inhibitors in the treatment of certain diseases, conditions, and/or disorders of the kidney according to the methods taught by Quigg, J Immunol 2003; 171:3319-24, the contents of which are herein incorporated by reference in their entirety.

Atypical Hemolytic Uremic Syndrome (aHUS)

Kidney-related indications may include atypical hemolytic uremic syndrome (aHUS). aHUS belongs to the spectrum of thrombotic microangiopathies. aHUS is a condition causing abnormal blood clots formation in small blood vessels of the kidneys. The condition is commonly characterized by hemolytic anemia, thrombocytopenia and kidney failure, and leads to end-stage renal disease (ESRD) in about half of all cases. aHUS has been associated with abnormalities of the alternative pathway of the complement system and may be caused by a genetic mutation in one of the genes that lead to increased activation of the alternative pathway. (Verhave et al., Nephrol Dial Transplant. 2014; 29 Suppl 4:iv131-41 and International Publication WO 2016/138520). aHUS may be treated by inhibitors that control the alternative pathway of complement activation, including C5 activation. In some embodiments, C5 inhibitor formulations of the present disclosure may be used to treat, prevent or delay development of aHUS. Methods and compositions for preventing and/or treating aHUS by complement inhibition may include any of those taught by Verhave et al. in Nephrol Dial Transplant. 2014; 29 Suppl 4:iv131-41 or International Publication WO 2016/138520, the contents of each of which are herein incorporated by reference in their entirety.

### Lupus Nephrilis

Kidney-related indications may include lupus nephritis. Lupus nephritis is a kidney inflammation caused by an autoimmune disease called systemic lupus erythematosus (SLE). Symptoms of lupus nephritis include high blood pressure; foamy urine; swelling of the legs, the feet, the hands, or the face; joint pain; muscle pain; fever; and rash. Lupus nephritis may be treated by inhibitors that control complement activity, including C5 inhibitor formulations of the present disclosure. Methods and compositions for preventing and/or treating Lupus nephritis by complement inhibition may include any of those taught in US publication No. US2013/0345257 or U.S. Pat. No. 8,377,437, the contents of each of which are herein incorporated by reference in their entirety.

### Membranous Glomerulonephritis (MGN)

Kidney-related indications may include membranous glomerulonephritis (MGN). MGN is a disorder of the kidney that may lead to inflammation and structural changes. MGN is caused by antibodies binding to a soluble antigen in kidney capillaries (glomerulus). MGN may affect kidney functions, such as filtering fluids and may lead to kidney failure. In some embodiments, C5 inhibitor formulations of the present disclosure may be used to prevent and/or treat MGN by inhibiting C5 activity. C5 inhibitor formulations may be used according to methods of preventing and/or treating MGN by complement inhibition taught in U.S. publication No. US2010/0015139 or in International publication No. WO2000/021559, the contents of each of which are herein incorporated by reference in their entirety.

### Hemodialysis Complications

Kidney-related indications may include hemodialysis complications. Hemodialysis is a medical procedure used to maintain kidney function in subjects with kidney failure. In hemodialysis, the removal of waste products such as creatinine, urea, and free water from blood is performed externally. A common complication of hemodialysis treatment is chronic inflammation caused by contact between blood and the dialysis membrane. Another common complication is thrombosis referring to a formation of blood clots that obstructs the blood circulation. Studies have suggested that these complications are related to complement activation. Hemodialysis may be combined with complement inhibitor therapy to provide means of controlling inflammatory responses and pathologies and/or preventing or treating thrombosis in subjects going through hemodialysis due to kidney failure. In some embodiments, C5 inhibitor formulations of the present disclosure may be used to prevent and/or treat complications associated with hemodialysis by inhibiting complement activation. Methods of using C5 inhibitor formulations for treatment of hemodialysis complications may be carried out according to any of the methods taught by DeAngelis et al in Immunobiology, 2012, 217(11): 1097-1105 or by Kourtzelis et al. Blood, 2010, 116(4):631-639, the contents of each of which are herein incorporated by reference in their entirety.

### Ocular Indications

Complement-related indications may include ocular indications. Ocular indications are therapeutic indications related to the eye. In a healthy eye the complement system is activated at a low level and is continuously regulated by membrane-bound and soluble intraocular proteins that protect against pathogens. Therefore, the activation of complement plays an important role in several complications related to the eye and controlling complement activation may be used to treat such diseases. In some embodiments, C5 inhibitor formulations of the present disclosure may be used to treat ocular indications by inhibiting complement activity. C5 inhibitor formulations may be used as complement inhibitors in the treatment of ocular disease according to any of the methods taught by Jha et al. in Mol Immunol. 2007; 44(16): 3901-3908 or in U.S. Pat. No. 8,753,625, the contents of each of which are herein incorporated by reference in their entirety.

### Age-Related Macular Degeneration (AMD)

Ocular indications may include age-related macular degeneration (AMID). AMD is a chronic ocular disease causing blurred central vision, blind spots in central vision, and/or eventual loss of central vision. Central vision affects ability to read, drive a vehicle and/or recognize faces. AMD is generally divided into two types, non-exudative (dry) and exudative (wet). Dry AMD refers to the deterioration of the macula which is the tissue in the center of the retina. Wet AMD refers to the failure of blood vessels under the retina leading to leaking of blood and fluid. Several human and animal studies have identified complement proteins that are related to AMD and novel therapeutic strategies included controlling complement activation pathways, as discussed by Jha et al. in Mol Immunol. 2007; 44(16): 3901-8. In some embodiments, C5 inhibitor formulations of the present disclosure may be used to prevent and/or treat AMD by inhibiting ocular complement activation. Methods of the present disclosure involving the use of C5 inhibitor formulations for prevention and/or treatment of AMD may include any of those taught in US publication Nos. US2011/0269807 or US2008/0269318, the contents of each of which are herein incorporated by reference in their entirety.

### Corneal Disease

Ocular indications may include corneal disease. The complement system plays an important role in protection of the cornea from pathogenic particles and/or inflammatory antigens. The cornea is the outermost front part of the eye covering and protecting the iris, pupil and anterior chamber and is therefore exposed to external factors. Corneal diseases include, but are not limited to, keratoconus, keratitis, ocular herpes and/or other diseases. Corneal complications may cause pain, blurred vision, tearing, redness, light sensitivity and/or corneal scarring. The complement system is critical for corneal protection, but complement activation may cause damage to the corneal tissue after an infection is cleared as certain complement compounds are heavily expressed. In some embodiments, C5 inhibitor formulations of the present disclosure may be used to prevent and/or treat corneal diseases by inhibiting ocular complement activation. Methods of the present disclosure for modulating complement activity in the treatment of corneal disease may include any of those taught by Jha et al. in Mol Immunol. 2007; 44(16): 3901-8, the contents of which are herein incorporated by reference in their entirety.

### Autoimmune Uveitis

Ocular indications may include autoimmune uveitis. Uvea is the pigmented area of the eye including the choroids, iris and ciliary body of the eye. Uveitis causes redness, blurred vision, pain, synechia and may eventually cause blindness. Studies have indicated that complement activation products are present in the eyes of patients with autoimmune uveitis and complement plays an important role in disease development. In some embodiments, C5 inhibitor formulations of the present disclosure may be used to treat and/or prevent uveitis. Such treatments may be carried out according to any of the methods identified in Jha et al. in Mol Immunol. 2007. 44(16): 3901-8, the contents of which are herein incorporated by reference in their entirety.

### Diabetic Retinopathy

Ocular indications may include diabetic retinopathy, which is a disease caused by changes in retinal blood vessels in diabetic patients. Retinopathy may cause blood vessel swelling and fluid leaking and/or growth of abnormal blood vessels. Diabetic retinopathy affects vision and may eventually lead to blindness. Studies have suggested that activation of complement has an important role in the development of diabetic retinopathy. In some embodiments, C5 inhibitor formulations of the present disclosure may be used to prevent and/or treat diabetic retinopathy. C5 inhibitor formulations may be used according to methods of diabetic retinopathy treatment described in Jha et al. Mol Immunol. 2007; 44(16): 3901-8, the contents of which are herein incorporated by reference in their entirety.

### Pregnancy-Related Indications

Complement-related indications may include pregnancy-related indications. Pregnancy-related indications are therapeutic indications involving child birth and/or pregnancy. Pregnancy-related indications may include pre-eclampsia and/or HELLP (abbreviation standing for syndrome features of 1) hemolysis, 2) elevated liver enzymes and 3) low platelet count) syndrome. Pre-eclampsia is a disorder of pregnancy with symptoms including elevated blood pressure, swelling, shortness of breath, kidney dysfunction, impaired liver function and/or low blood platelet count. Pre-eclampsia is typically diagnosed by a high urine protein level and high blood pressure. HELLP syndrome is a combination of hemolysis, elevated liver enzymes and low platelet conditions. Hemolysis is a disease involving rupturing of red blood cells leading to the release of hemoglobin from red blood cells. Elevated liver enzymes may indicate a pregnancy-induced liver condition. Low platelet levels lead to reduced clotting capability, causing danger of excessive bleeding. HELLP is associated with a pre-eclampsia and liver disorder. HELLP syndrome typically occurs during the later stages of pregnancy or after childbirth. It is typically diagnosed by blood tests indicating the presence of the three conditions it involves. Typically HELLP is treated by inducing delivery.

Studies suggest that complement activation occurs during HELLP syndrome and pre-eclampsia and that certain complement components are present at increased levels during HELLP and pre-eclampsia. Complement inhibitor formulations of the present disclosure may be used as therapeutic agents to prevent and/or treat these and other pregnancy-related conditions. C5 inhibitor formulations may be used according to methods of preventing and/or treating HELLP and pre-eclampsia taught by Heager et al. in Obstetrics & Gynecology, 1992, 79(1): 19-26 or in International publication No. WO2014/078622, the contents of each of which are herein incorporated by reference in their entirety.

### Dosage and Administration

Administration of formulations presented herein may be achieved in a number of different ways, including any route that results in a therapeutically effective outcome. These administration routes include, but are not limited to enteral, gastroenteral, epidural, oral, peridural, intracerebral (into the cerebrum), intratracheal (into the airways for delivery to the lung), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal (IVT, into the posterior chamber of the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), buccal, sublingual, sublabial, enema, eye drops (onto the conjunctiva), or in ear drops.

Local delivery avoids gut permeability and systemic exposure. For example, formulations may be used in the posterior section of the eye by direct injection. They may be applied in the gut to target enzymes. They may be used topically in dermatologic applications (e.g., creams, ointments, transdermal patches).

In some embodiments, sustained release formulations are administered parenterally. Parenteral administration may include, but is not limited to intravitreal, intrathecal, subdural, epidural, intraperitoneal, intramuscular, subcutaneous, and intradermal administration. To inhibit complement activity, the sustained release formulations may include a complement inhibitor as a therapeutic agent. The complement inhibitor may be R5000.

In some embodiments, methods of treating complement-related indications according to the present disclosure include administering a sustained release formulation that includes R5000 as a therapeutic agent. R5000 may be included in the formulation at a concentration of from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 20 mg/mL, from about 5 mg/mL to about 30 mg/mL, from about 10 mg/mL to about 40 mg/mL, from about 15 mg/mL to about 50 mg/mL, from about 20 mg/mL to about 75 mg/mL, from about 25 mg/mL to about 100 mg/mL, from about 30 mg/mL to about 125 mg/mL, from about 35 mg/mL to about 150 mg/mL, from about 40 mg/mL to about 175 mg/mL, from about 45 mg/mL to about 200 mg/mL, from about 50 mg/mL to about 225 mg/mL, from about 60 mg/mL to about 250 mg/mL, from about 70 mg/mL to about 300 mg/mL, from about 80 mg/mL to about 350 mg/mL, from about 90 mg/mL to about 400 mg/mL, from about 100 mg/mL to about 450 mg/mL, from about 110 mg/mL to about 500 mg/mL, from about 120 mg/mL to about 600 mg/mL, from about 130 mg/mL to about 700 mg/mL, from about 140 mg/mL to about 800 mg/mL, from about 150 mg/mL to about 900 mg/mL, from about 200 mg/mL to about 1000 mg/mL, or more than 1000 mg/ml. In some embodiments, sustained release formulations include R5000 at a concentration of about 130 mg/ml. Formulations may be administered at a dose sufficient to provide from about 0.01 mg/kg to about 1.0 mg/kg, from about 0.02 mg/kg to about 2.0 mg/kg, from about 0.05 mg/kg to about 3.0 mg/kg, from about 0.10 mg/kg to about 4.0 mg/kg, from about 0.15 mg/kg to about 4.5 mg/kg, from about 0.20 mg/kg to about 5.0 mg/kg, from about 0.30 mg/kg to about 7.5 mg/kg, from about 0.40 mg/kg to about 10 mg/kg, from about 0.50 mg/kg to about 12.5 mg/kg, from about 1.0 mg/kg to about 15 mg/kg, from about 2.0 mg/kg to about 20 mg/kg, from about 5.0 mg/kg to about 25 mg/kg, from about 10 mg/kg to about 45 mg/kg, from about 20 mg/kg to about 55 mg/kg, from about 30 mg/kg to about 65 mg/kg, from about 40 mg/kg to about 75 mg/kg, from about 50 mg/kg to about 150 mg/kg, from about 100 mg/kg to about 250 mg/kg, from about 200 mg/kg to about 350 mg/kg, from about 300 mg/kg to about 450 mg/kg, from about 400 mg/kg to about 550 mg/kg, or from about 500 mg/kg to about 1000 mg/kg of R5000.

### Kits and Devices

In some embodiments, the present disclosure provides kits for administration of formulations presented herein or kits for the preparation of formulations presented herein. Some kits include a container. Some kits may include at least one vial, test tube, flask, bottle, syringe and/or other containers, into which compounds and/or formulations are placed, preferably, suitably allocated. Kits may also include containers with sterile, pharmaceutically acceptable buffer and/or other diluent.

Kits may include instructions for employing the kit components as well the use of any other reagent not included in the kit. Instructions may include variations that can be implemented.

While various embodiments of the invention have been particularly shown and described, it will be understood by those skilled in the art that various changes in form and details may be made therein without departing from the spirit and scope of the invention as defined by the appended claims.

### Definitions

Biological system: As used herein, the term “biological system” refers to a cell, a group of cells, a tissue, an organ, a group of organs, an organelle, a biological signaling pathway (e.g., a receptor-activated signaling pathway, a charge-activated signaling pathway, a metabolic pathway, a cellular signaling pathway, etc.), a group of proteins, a group of nucleic acids, or a group of molecules (including, but not limited to biomolecules) that carry out at least one biological function or biological task within cellular membranes, cellular compartments, cells, cell cultures, tissues, organs, organ systems, organisms, multicellular organisms, or any biological entities. In some embodiments, biological systems are cell signaling pathways comprising intracellular and/or extracellular signaling biomolecules. In some embodiments, biological systems comprise proteolytic cascades (e.g., the complement cascade).

Control system: As used herein, the term “control system” refers to a biological system that is untreated and used for comparison to a biological system that is or has been treated or otherwise manipulated.

Downstream event: As used herein, the term “downstream” or “downstream event,” refers to any event occurring after and as a result of another event. In some cases, downstream events are events occurring after and as a result of C5 cleavage and/or complement activation. Such events may include, but are not limited to, generation of C5 cleavage products, activation of MAC, hemolysis, and hemolysis-related disease (e.g., PNH).

Pharmaceutical composition: As used herein, the term “pharmaceutical composition” refers to a composition having at least one active ingredient (e.g., an inhibitor polypeptide) in a form and amount that permits the active ingredient to be therapeutically effective.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Subject: As used herein, the term “subject” refers to any organism to which a compound in accordance with the invention may be administered, e.g., for experimental, diagnostic, prophylactic, and/or therapeutic purposes. Typical subjects include animals (e.g., mammals such as mice, rats, rabbits, porcine subjects, non-human primates, and humans).

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Preparation of R5000

R5000 was prepared essentially as described in International Publication No. WO2017/105939. Polypeptides were synthesized using standard solid-phase Fmoc/tBu methods. The synthesis was performed on a Liberty automated microwave peptide synthesizer (CEM, Matthews N.C.) using standard protocols with Rink amide resin, although other automated synthesizers without microwave capability may also be used. All amino acids were obtained from commercial sources. The coupling reagent used was 2-(6-chloro-1-H-benzotriazole-1yl)-1,1,3,3,-tetramethylaminium hexafluorophosphate (HCTU) and the base was diisopropylethylamine (DIEA). Polypeptides were cleaved from resin with 95% TFA, 2.5% TIS and 2.5% water for 3 hours and isolated by precipitation with ether. The crude polypeptides were purified on a reverse phase preparative HPLC using a C18 column, with an acetonitrile/water 0.1% TFA gradient from 20%-50% over 30 min. Fractions containing pure polypeptides were collected and lyophilized and all polypeptides were analyzed by LC-MS.

R5000 was prepared as a cyclic peptide containing 15 amino acids (4 of which are unnatural amino acids), an acetylated N-terminus, and a C-terminal carboxylic acid. The C-terminal lysine of the core peptide has a modified side chain, forming a N-ε-(PEG24-γ-glutamic acid-N-α-hexadecanoyl) lysine reside. This modified side chain includes a polyethyleneglycol spacer (PEG24) attached to an L-γ glutamic acid residue that is derivatized with a palmitoyl group. The cyclization of R5000 is via a lactam bridge between the side-chains of Lys1 and Asp6. All of the amino acids in R5000 are L-amino acids. R5000 has a molecular weight of 3562.23 g/mol and a chemical formula of C₁₇₂H₂₇₈N₂₄O₅₅.

R5000 blocks the proteolytic cleavage of C5 into C5a and C5b. R5000 can also bind to C5b and block C6 binding which prevents the subsequent assembly of the MAC.

### Example 2. Formulations for Sustained Release

Formulations including R5000 dissolved in various lipid excipients capable of forming in-situ liquid crystal (LC) phase depots were screened for sustained release of R5000. The assessment was performed by conducting in-vitro release studies as well as in-vivo pharmacokinetic (PK) studies in rats and PK and pharmacodynamic (PD) studies in monkeys using these formulations. In total, 10 formulations (about one formulation from each of the evaluated systems) were selected for dose range finding (DRF) studies in rats based on their performance.

Formulations were prepared by first weighing an appropriate amount of R5000 in vials. Appropriate volumes of these glycerides (see Table 2) were then added to the vials containing the drug.

TABLE 2
Glycerides of fatty acids

| | Glyceride Percentage | | | Chain | Double |
|---|---|---|---|---|---|
| Name | Mono- | Di- | Tri- | Length | Bonds |
| PECEOL ™ | 44 | 45 | 10 | 18 | 1 |
| MAISINE ® 35-1 | 41 | 47 | 11 | 18 | 2 |
| MAISINE ® CC | 35 | 52 | 13 | 18 | 2 |
| Monoolein (MC18-1) | 100 | 0 | 0 | 18 | 1 |
| Monolinolein (MC18-2) | 100 | 0 | 0 | 18 | 2 |
| Dilinolein (DC18-2) | 0 | 100 | 0 | 18 | 2 |
| Trilinolein (TC18-2) | 0 | 0 | 100 | 18 | 2 |
| Monolinolenin (MC18-3) | 100 | 0 | 0 | 18 | 3 |
| Dilinolenin (DC18-3) | 0 | 100 | 0 | 18 | 3 |
| Trilinolenin (TC18-3) | 0 | 0 | 100 | 18 | 3 |
| Dicaprylin (DC8-0) | 0 | 100 | 0 | 8 | 0 |
| Tricaprylin (TC8-0) | 0 | 0 | 100 | 8 | 0 |
| Dicaprin (DC10-0) | 0 | 100 | 0 | 10 | 0 |
| Tricaprin (TC10-0) | 0 | 0 | 100 | 10 | 0 |

Glycerides that are solid at room temperature were melted by keeping them in an incubator at 50° C. for 15 minutes, whereas glycerides that are liquid at room temperature were used as is. Similarly, polyethylene glycol 300 (PEG-300), propylene glycol (PG), and polysorbate (PS; Croda, Inc., Edison, N.J.) excipients, that are liquid at room temperature, were measured to the required volume and added to the vials. Other excipients that are solid at room temperature and cannot be melted at 50° C. such as phosphatidylcholine (PC) head-group lipids [e.g., diglycerophosphocholine with 8 carbon chain length (DC8-0PC; Avanti Polar, Alabaster, Ala.) or diglycerophosphocholine with 10 carbon chain length (DC10-0PC; Avanti Polar, Alabaster, Ala.)], poloxamer 407 (POL407; Sigma-Aldrich, St. Louis, Mo.), and sodium deoxycholate (Na-DC; Sigma-Aldrich, St. Louis, Mo.) were weighed to required weight and added to these vials. These vials were then kept in an incubator at 50° C. for about 1 hour and vortexed intermittently for 2-3 times in order to completely dissolve the components and generate a clear transparent solution. These formulations were then used for in-vitro or in-vivo studies.

For release studies, phosphate buffered saline (PBS) (150 ml each) was taken in glass jars containing stir bars and kept on a stir plate in an incubator maintained at 34° C. for about 2 hours at a consistent stir rate. R5000 formulations (100 μl each) were then added carefully in the center of the jar on top of the PBS using positive displacement pipetting. Samples of PBS (0.5 ml) were collected one inch away from the formulation blob spinning in the center after specified time intervals and the jar was replaced with 0.5 ml of fresh PBS. PBS (350 μl) was then aliquoted from the 0.5 ml PBS samples that were collected after specified time intervals and placed in 96-well plates. UV absorbance of these aliquoted samples was measured at 285, 370, and 390 nm using a SPECTRAMAX® M3 (Molecular Devices, Sunnyvale, Calif.) detector using PBS as a blank. The absorbance measured at 285 nm was then corrected by Rayleigh Scattering Equation as follows. The percent drug released was then calculated using a calibration curve generated by measuring the absorbance of standard solutions of R5000 at 285 nm.

$${A}_{285 Corrected} = {A}_{285 Measured} - ( \frac{{A}_{370 Measured} - {A}_{390 Measured}}{{370}^{- 4} - {390}^{- 4}} ) {285}^{- 4} - {A}_{370 Measured} + ( \frac{{A}_{370 Measured} - {A}_{390 Measured}}{{285}^{- 4}} ) {370}^{- 4}$$

To measure pharmacokinetics (PK) in rats, male Sprague-Dawley rats with a single jugular vein cannula (JVC) were used. Animals were allowed to acclimate to the test facility for at least 2 days prior to study start. Formulations were kept at room temp until ready for dosing and then incubated at 37° C. for at least 20 minutes before injection. Formulations were administered subcutaneously at 30 mg/kg dose ratio and the dosing syringes were weighed prior to and immediately following dose administration to gravimetrically determine the amount of dose delivered. Serial blood samples were collected via jugular vein cannula (JVC). If patency was lost, samples were obtained via jugular vein or tail vein. Blood samples were collected into K2EDTA tubes and stored on wet ice until processed to plasma by centrifugation (3500 rpm at 5° C.) within 30 minutes of collection. All plasma samples were transferred into separate 96-well plates (matrix tubes) and stored at −80° C. until R5000 concentration analysis via liquid chromatography (LC) tandem mass spectrometry (MS/MS) using a residual gas analysis (RGA) 1 assay.

For PK and pharmacodynamic (PD) analysis in monkeys, non-naive Cynomolgus monkeys (2 to 4 kg at the time of dosing) were used. R5000 formulations were kept at room temperature until ready for dosing and then incubated at 37° C. for at least 20 minutes before injection. Formulations were administered subcutaneously on the back of each animal at a previously specified (2, 4, and 8) mg/kg dose ratio. Whole blood samples (1.5 ml each) were collected from a peripheral vessel after specified time intervals. Cephalic or saphenous vessels were used when femoral collection was unsuccessful. Whole blood samples were collected into K2EDTA tubes for plasma processing and were kept on wet ice before processing. Blood was centrifuged for 10±2 minutes in a refrigerated centrifuge. Plasma samples were then distributed into ˜0.1 mL aliquots with up to 4 total aliquots of ˜0.1 mL. Any remaining plasma was placed in aliquot 5. Plasma samples were then placed in tubes and stored frozen at −70° C.±10° C. until analysis. R5000 concentration analysis was performed via LC/MS/MS using an RGA 1 assay.

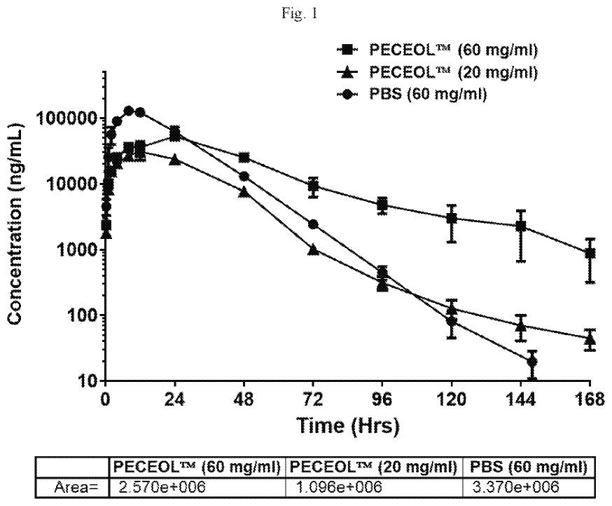

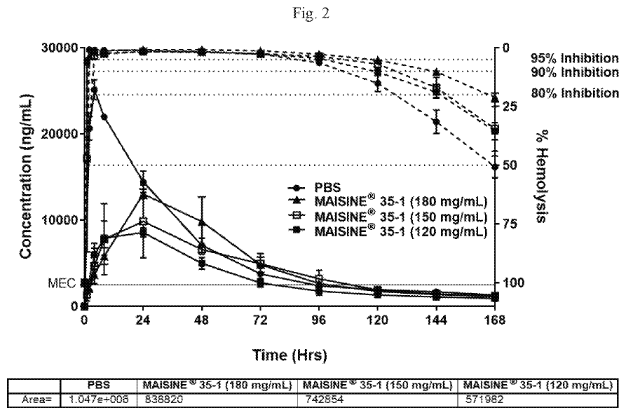

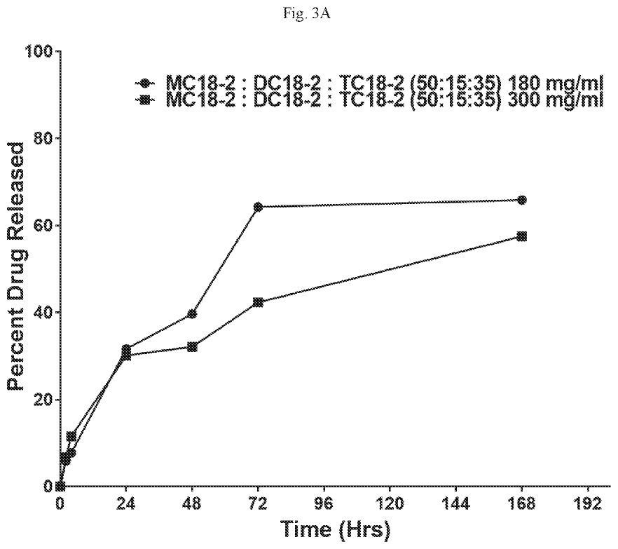

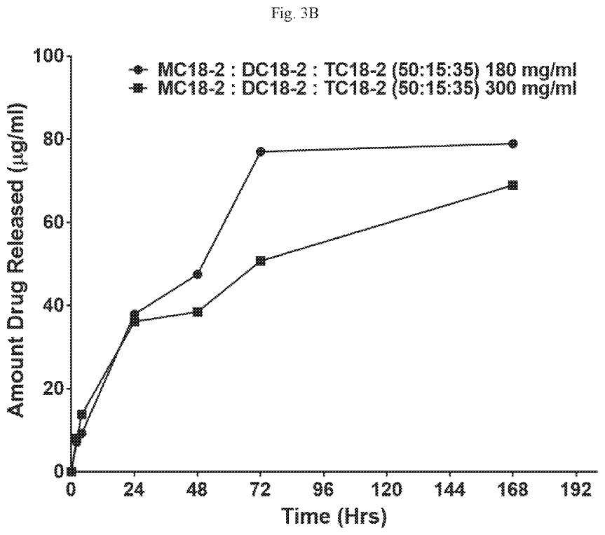

Higher area under the curve (AUC) as well as sustained release was obtained in rats at a 30 mg/kg dose by increasing the R5000 concentration from 20 to 60 mg/ml in PECEOL™ (Gattefosse Pharmaceuticals, Paramus, N.J.) formulation (see FIG. 1). Similar results were obtained in monkeys at a 4 mg/kg dose by gradually increasing the R5000 concentration from 120 to 150 and then to 180 mg/ml in MAISINE® 35-1 (Gattefosse Pharmaceuticals, Paramus, N.J.) formulation (see FIG. 2). The AUC was increased with an increase in concentration, which also enhanced the PD coverage in monkeys. These results can be attributed to a higher concentration gradient maintained over a longer duration across a lower volume of the formulation liquid crystal matrix that assisted in complete recovery. However, further increases in R5000 concentration above 180 mg/ml increased the viscosity of the formulations as well as reduced the recovery. For example, increasing the concentration from 180 to 300 mg/ml in MC18-2:DC18-2:TC18-2 (50:15:35) formulation significantly reduced the recovery during in-vitro assay (see FIG. 3A and FIG. 3B). These effects may be due to changes in the nature of LC phase at very high concentrations.

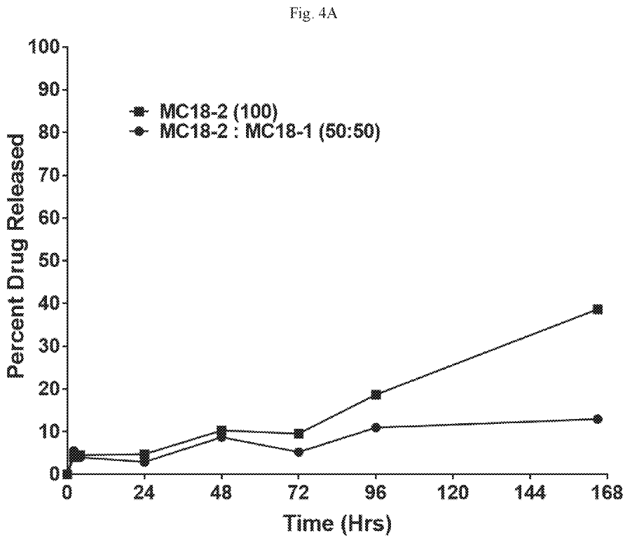

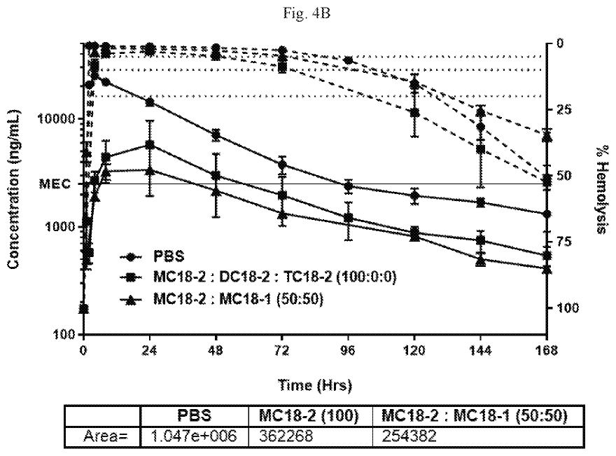

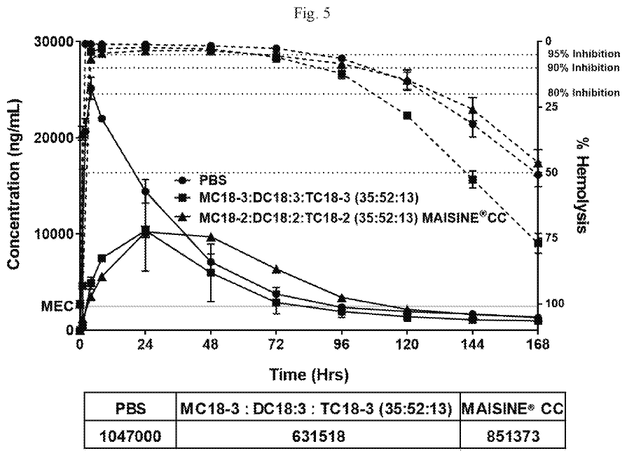

When glycerides containing fatty acid chains with 2 double bonds were used rather than those with 1 double bond, the recovery in vitro and AUC in monkeys was increased (FIG. 4A and FIG. 4B, respectively). However, increasing the unsaturation further by using glycerides containing fatty acid chains with 3 double bonds resulted in reduction of AUC as shown in FIG. 5. Further, the formulation depicted signs of degradation such as coloration and odor, which could be attributed to lower stability with further increase in unsaturation.

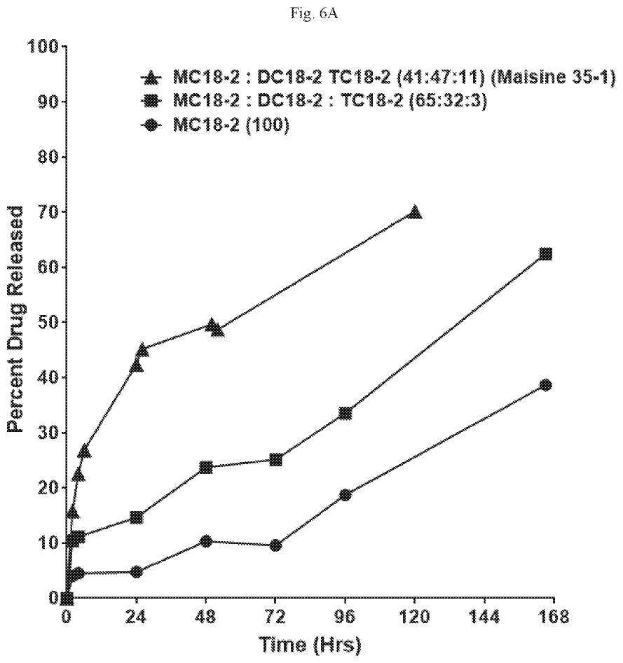

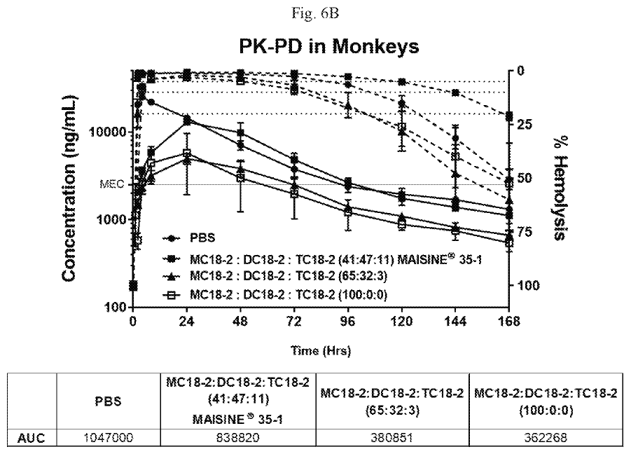

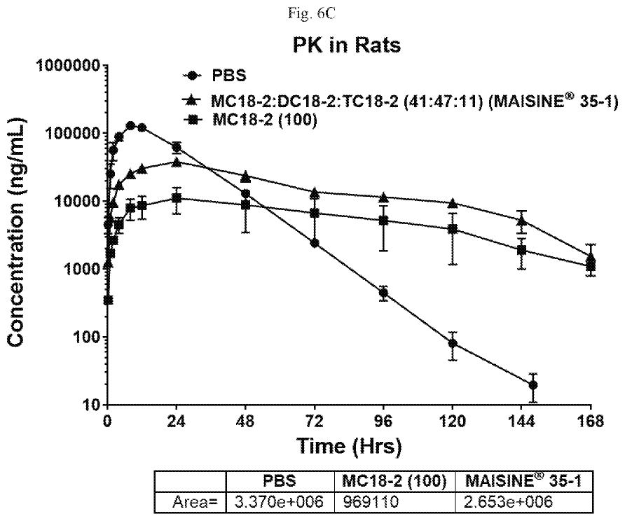

As shown in FIG. 6A, the release rate of R5000 in vitro was increased by lowering the mono-glyceride content of the formulations, which also enhanced the recovery of R5000. At about 40% mono-glyceride content, the initial release rate (up to 48 hours) was higher than at later time points. These data correlated well with in vivo PK-PD in monkeys (FIG. 6B) and PK in rats (FIG. 6C), where the AUC increased with a decrease in mono-glyceride content. The PD coverage in monkeys was better for formulations containing 40% mono-glyceride that also exhibited the highest AUC. Although the AUC and PD coverage was better for formulations having lower mono-glyceride content (40%), it was necessary to reserve R5000 for later days in a week by keeping initial release rate lower (up to 48 hours) and increasing the recovery over later time points. Accordingly, higher mono-glyceride formulations were prepared and R5000 recovery was increased by optimizing di- and tri-glycerides levels and by including other excipients in the formulations.

To improve R5000 recovery in formulations with higher mono-glyceride content, while maintaining lower initial release, additional formulations were tested using di- and tri-glycerides with shorter chain lengths (e.g., DC8-0, TC8-0, DC10-0, and TC10-0). In vitro release testing was used to select top performing formulations out of many tested. Formulations selected for further testing included MC18-2:DC8-0:TC8-0 (70:20:10) and MC18-2:DC10-0:TC10-0 (60:20:20).

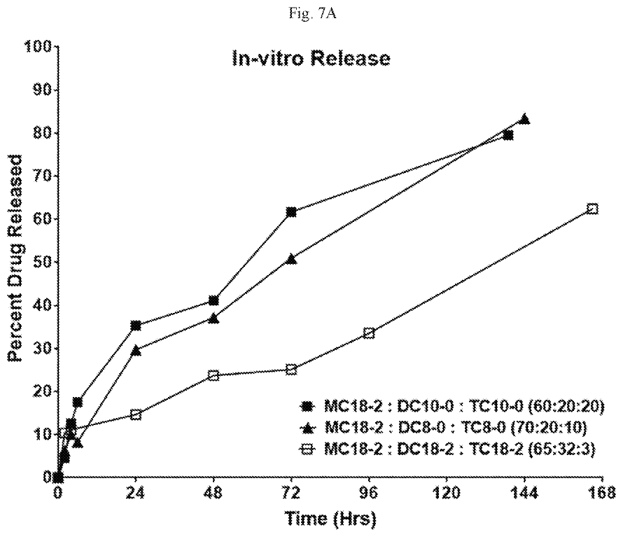

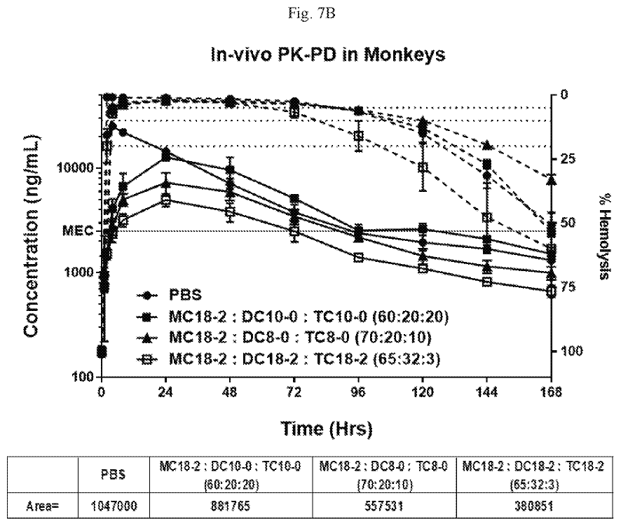

In general, the recovery was improved by using shorter chain di- and tri-glycerides at higher mono-glyceride content. As shown in FIG. 7A, using di- and tri-glycerides of C8-0 and C10-0 at relatively higher mono-glyceride ratios of 70 and 60 increased the recovery during in-vitro release study as compared to that using di- and tri-glycerides of C18-2 at a higher mono-glyceride ratio of 65. Further, these data correlated well with the PK study in monkeys (FIG. 7B). The rank order of formulations in achieving higher AUC in monkeys was the same as those yielding higher recovery during the in vitro study. Top formulations were selected for DRF studies in rats. In rat release and tolerability studies, MC18-2:DC10-0:TC10-0 (60:20:20) administration did not result in any mass or scab formation and no signs of inflammation were observed.

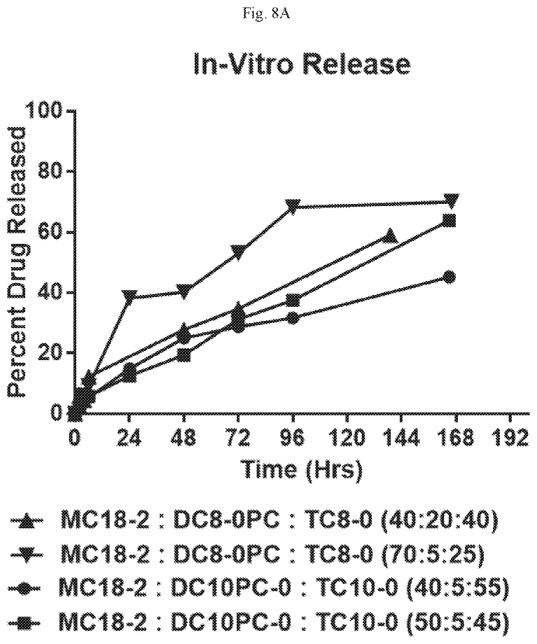

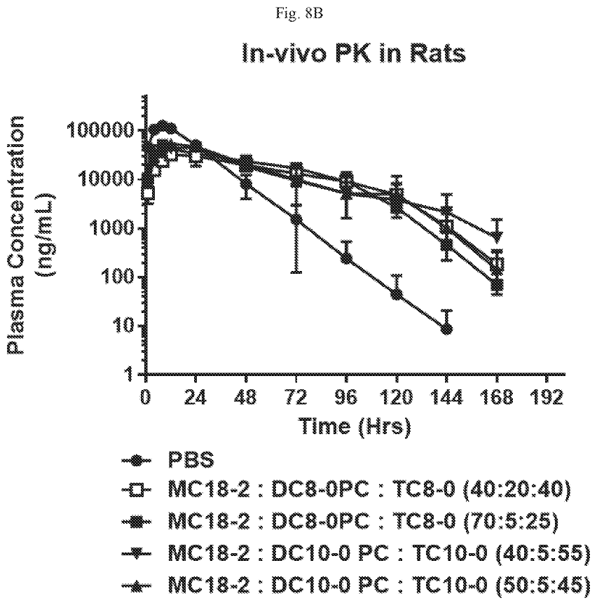

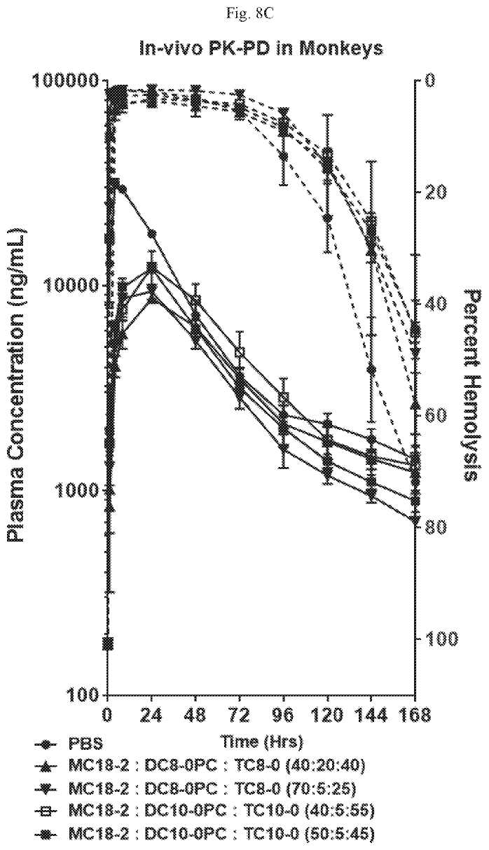

The impact of using different head-groups was also assessed. Diglycerophosphocholine head-groups with C8-0 and C10-0 chains were used. Several combinations were explored by in-vitro release testing and the top two formulations were selected from each system. MC18-2:DC8-0PC:TC8-0PC (40:20:20); MC18-2:DC8-0PC:TC8-0PC (70:5:25); MC18-2:DC10-0PC:TC10-0PC (40:5:55); and MC18-2:DC10-PC:TC10-0PC (50:5:45) were selected. In general, the inclusion of Di-PC lipids lowered the initial release (0-96 hrs) during in vitro study (FIG. 8A) with a slightly better recovery at later time points (120-168 hrs). However, not much benefit was detected in vivo (see FIG. 8B for rat PK and FIG. 8C for monkey PK-PD).

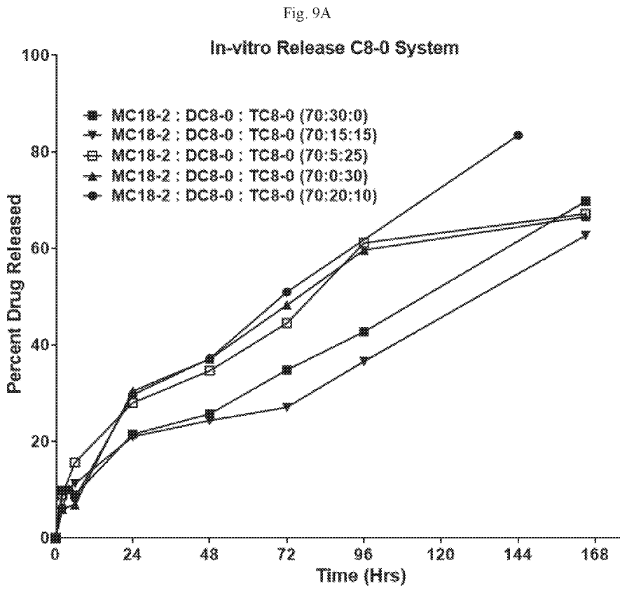

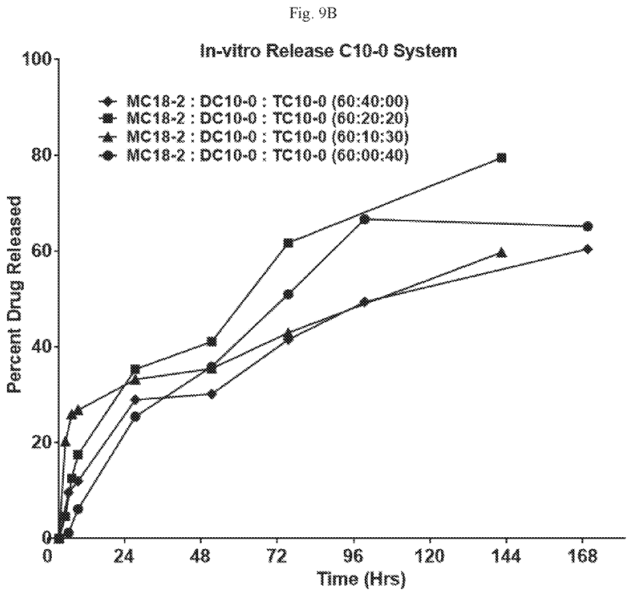

Varying the ratio of di- and tri-glycerides had a significant impact on the release rate in vitro. As shown in FIG. 9A and FIG. 9B, both C8-0 and C10-0 systems showed variable release rates at the same mono-glyceride content of 70% and 60%, respectively. As described earlier, the top two formulations from each of these systems exhibiting relatively lower initial release rate and the highest recovery in vitro were selected for DRF study in rats.

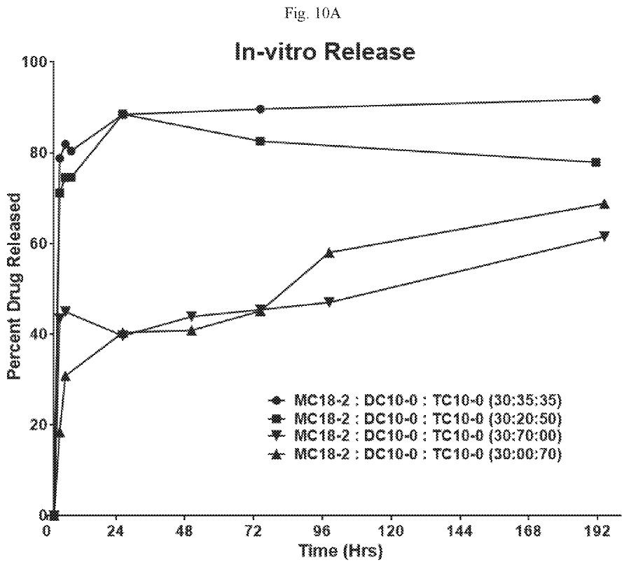

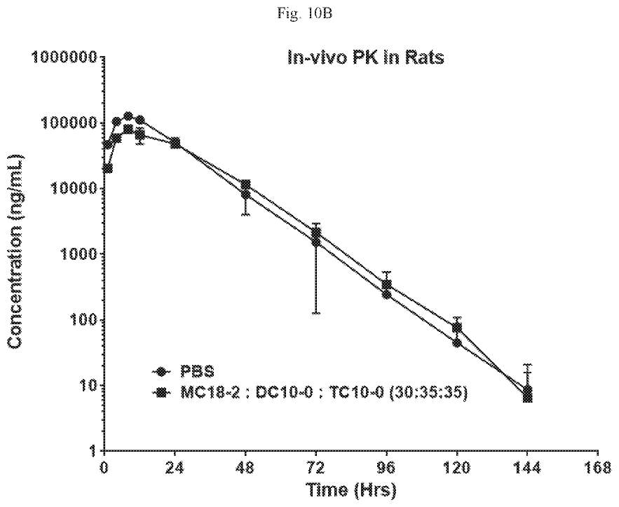

Variation in di- and tri-glyceride content drastically affected the release rate at lower mono-glyceride content (30%) for C10-0 systems. As shown in FIG. 10A, higher burst and lower recovery was detected for formulations containing either di- or tri-glycerides. Whereas, using both di- and tri-glycerides released almost all R5000 in only 6 hours. One of the formulations containing a M:D:T ratio of 30:35:35 from this system was tested in rats, which correlated well with the in vitro results showing an almost superimposable profile to that of PBS (FIG. 10B).

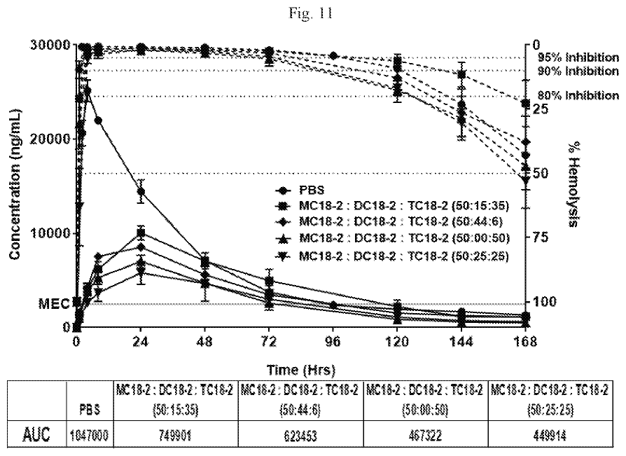

LC formulations containing only C18-2 lipids also showed significantly different PK profiles in monkeys with different AUCs (FIG. 11). The PK results correlated well with the PD data, where LC formulation MC18-2:DC18-2:TC18-2 (50:15:35) showed higher AUC as well as PD coverage for longer duration as compared to other formulations. Accordingly, this formulation was selected for further study in rats.

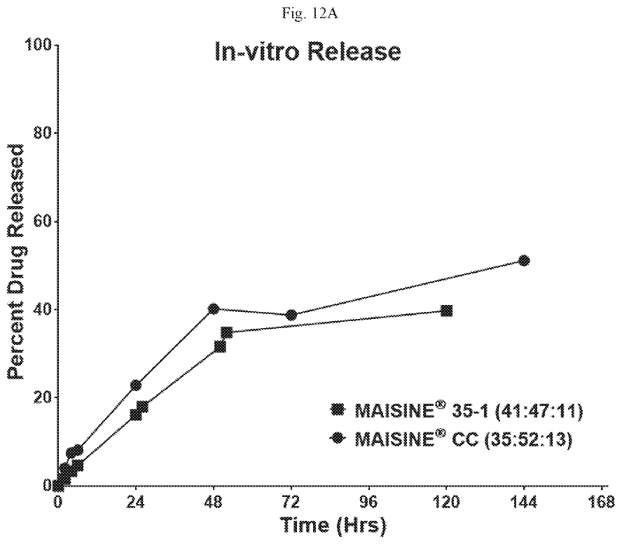

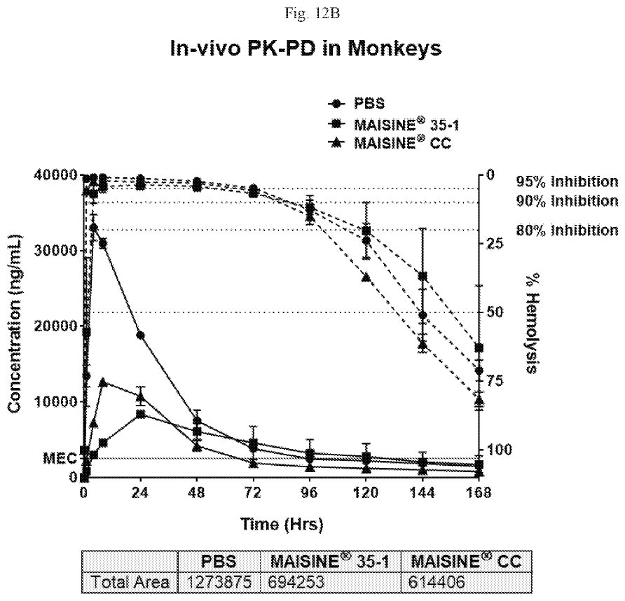

The ammonium salt of R5000 has lower solubility in water than the sodium salt and was tested in formulations with lower mono-glyceride contents of 35% and 40% to check for constant PK levels above minimum effective concentration. However, very low recovery was obtained for the ammonium salt in in vitro release (FIG. 12A). These data correlated well with the in vivo PK in monkeys (FIG. 12B).

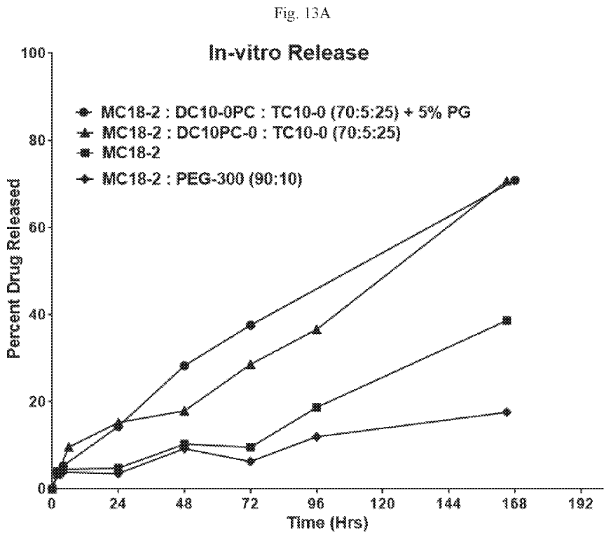

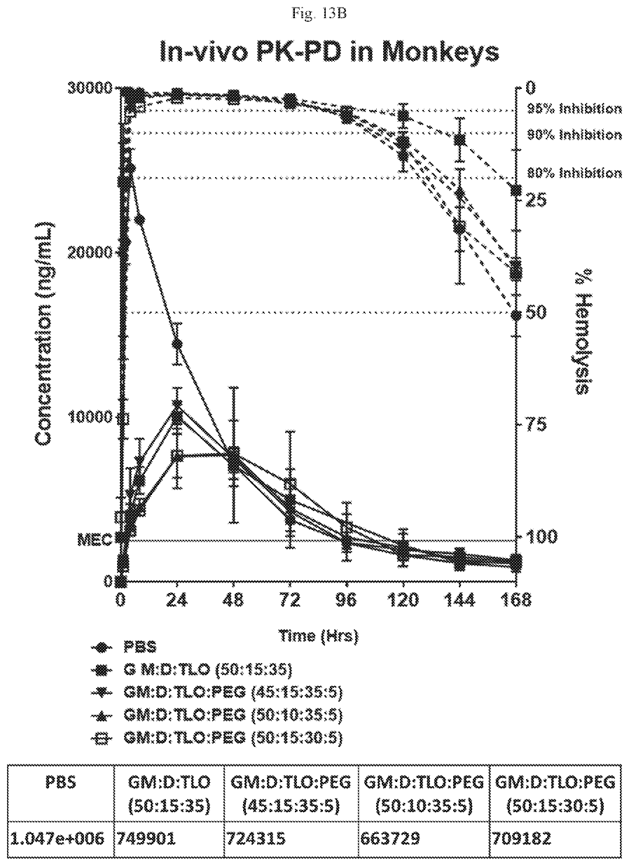

As shown in FIG. 13A, addition of PEG-300 reduced the recovery of R5000 for the MC18-2 formulation, whereas addition of PG increased the recovery for MC18-2:DC10-0PC:TC10-0 (70:5:25) formulation. Addition of PEG-300 to the MC18-2:DC18-2:TC18-2 formulation didn't show any improvement in AUC in monkeys (FIG. 13B). Addition of surfactants had a variable effect on different systems.

### Example 3. Transition of the Formulation Composition to Non-Lamellar Liquid Crystalline Phase

Polarized light microscopy was used to observe the transition of formulations to a non-lamellar liquid crystalline structure after exposure to an aqueous buffer. R5000 was dissolved at 180 mg/ml concentration in a mixture of mono-, di-, and tri-glycerides of linoleic acid at a ratio of 41:47.1:11.3, respectively. This formulation was then exposed to excess PBS and observed by polarized light microscopy. Observations indicated that the formulation presented a birefringence of lamellar phase once exposed to PBS. This effect gradually disappeared because of the transition of the formulation to a non-lamellar liquid crystalline structure. The transition was observed within 90 minutes from the exposure to PBS.

1. A sustained release formulation comprising:
a therapeutic agent; and
a mixture of acylglycerols, said mixture comprising:
from about 40% to about 70% monoglycerides;
from about 0% to about 60% diglycerides; and
from about 0% to about 60% triglycerides.

2. The formulation of claim 1, wherein at least one acylglycerol from the mixture of acylglycerols comprises at least one fatty acid selected from one or more of a long chain fatty acid and a medium chain fatty acid.

3. The formulation of claim 1, wherein at least one acylglycerol from the mixture of acylglycerols comprises an unsaturated fatty acid.

4. The formulation of claim 1, wherein at least one acylglycerol from the mixture of acylglycerols comprises a saturated fatty acid.

5. The formulation of claim 2, wherein at least one acylglycerol from the mixture of acylglycerols comprises a long chain fatty acid selected from one or more of linoleic acid and oleic acid.

6. The formulation of claim 2, wherein at least one acylglycerol from the mixture of acylglycerols comprises a medium chain fatty acid selected from one or more of capric acid and caprylic acid.

7. The formulation of claim 1, wherein the mixture of acylglycerols comprises about 60% monoglycerides.

8. The formulation of claim 7, wherein said monoglycerides comprise linoleic acid.

9. The formulation of claim 1, wherein the mixture of acylglycerols comprises about 20% diglycerides.

10. The formulation of claim 1, wherein the mixture of acylglycerols comprises less than 20% diglycerides.

11. The formulation of claim 9 or 10, wherein said diglycerides comprise capric acid.

12. The formulation of claim 1, wherein the mixture of acylglycerols comprises about 20% triglycerides.

13. The formulation of claim 1, wherein the mixture of acylglycerols comprises less than 20% triglycerides.

14. The formulation of claim 12 or 13, wherein said triglycerides comprise capric acid.

15. The formulation of claim 1, wherein the mixture of acylglycerols comprises about 60% monolinolein.

16. The formulation of claim 15, wherein the mixture of acylglycerols comprises about 20% dicaprylin.

17. The formulation of claim 15 or 16, wherein the mixture of acylglycerols comprises about 20% tricaprylin.

18. The formulation of claim 1 comprising at least one excipient.

19. The formulation of claim 18, wherein the at least one excipient is selected from one or more of phosphate buffered saline and sodium deoxycholate.

20. The formulation of claim 1, wherein said formulation comprises propylene glycol.

21. The formulation of claim 1, wherein said formulation comprises a surfactant.

22. The formulation of claim 1, wherein the therapeutic agent is a peptide or peptidomimetic.

23. The formulation of claim 1, wherein the therapeutic agent is a complement inhibitor.

24. The formulation of claim 23, wherein the complement inhibitor is a C5 inhibitor.

25. The formulation of claim 24, wherein the C5 inhibitor is R5000.

26. A method of delivering a therapeutic agent to a subject comprising the steps of:
preparing the formulation of claim 1, and
administering the formulation to the subject.

27. The method of claim 26, wherein the formulation is a low viscosity formulation prior to administration.

28. The method of claim 27, wherein the formulation becomes highly viscous upon administration to the subject.

29. The method of claim 28, wherein the formulation forms a highly viscous non-lamellar liquid crystalline phase upon administration to the subject.

30. The method of claim 29, wherein the formulation becomes highly viscous upon contact with an aqueous bodily fluid.

31. The method of claim 30, wherein the therapeutic agent is continuously released from the formulation over an extended period of time after administration.

32. The method of claim 31, wherein the formulation is administered parenterally.

33. The method of claim 32, wherein the formulation is administered intravitreally, intrathecally, subdurally, epidurally, intraperitoneally, intramuscularly, subcutaneously, or intradermally.

34. A method of inhibiting complement activity in a subject, said method comprising administering the formulation of claim 25 to the subject.

35. The method of claim 34, wherein R5000 is present in the formulation at a concentration of from about 10 mg/ml to about 500 mg/ml.

36. The method of claim 34 or 35, wherein the formulation is administered to the subject at a dose sufficient to provide from about 0.01 mg/kg to about 500 mg/kg of R5000.

37. A method of treating a complement-related indication in a subject, said method comprising administering the formulation of claim 25 to the subject.

38. The method of claim 37, wherein R5000 is present in the formulation at a concentration of from about 10 mg/ml to about 500 mg/ml.

39. The method of claim 37 or 38, wherein the formulation is administered to the subject at a dose sufficient to provide from about 0.01 mg/kg to about 500 mg/kg of R5000.


SEQUENCE LISTING (1 sequence)

SEQ ID NO: 1 - protein, 15 aa, Artificial Sequence
  source: /note=“Description of Artificial Sequence Synthetic peptide”
  N-term Ac
  MISC_FEATURE (1)..(6): /note=“Lactam bridge between residues”
  MOD_RES (7)..(7): N-methyl-aspartic acid
  MOD_RES (8)..(8): Tert-butylglycine
  MOD_RES (10)..(10): 7-azatryptophan
  MOD_RES (14)..(14): Cyclohexylglycine
  MOD_RES (15)..(15): N-epsilon-(PEG24-gamma-glutamic acid-N-alpha- hexadecanoyl)Lys
KVERFDXXYXEYPXK